(19) United States
(12) Patent Application Publication
(10) Pub. No.: US 2015/0004219 A1
(43) Pub. Date: Jan. 1, 2015

(54) STABLE LIPOSOMES FOR DRUG DELIVERY

(71) Applicant: Yissum Research Development Company of the Hebrew University of Jerusalem, Ltd., Jerusalem (IL)

(72) Inventors: Yechezkel Barenholz, Jerusalem (IL); Tal Berman, Rishon LeZion (IL); Doron Friedman, Carmei Yosef (IL)

(21) Appl. No.: 14/375,877

(22) PCT Filed: Feb. 3, 2013
(86) PCT No.: PCT/IL2013/050100

§ 371 Date: Jul. 31, 2014

Related U.S. Application Data
(60) Provisional application No. 61/594,090, filed on Feb. 2, 2012.

(51) Int. Cl. A61K 47/20; A61K 31/4745; A61K 31/704; A61K 9/127; A61K 45/06

(52) U.S. Cl. CPC A61K 47/20; A61K 9/127; A61K 45/06; A61K 31/704; A61K 31/4745; A61K 9/1278
USPC 424/450; 514/34; 514/283

(57) ABSTRACT

Liposomes with an entrapped amphipathic weak base and alkyl or aryl sulfonate are described as well as methods of making and using these liposomes.

### CROSS-REFERENCE TO RELATED APPLICATIONS

The present application claims the benefit of U.S. Provisional Application No. 61/594,090, filed Feb. 2, 2012, the disclosure of which is hereby incorporated by reference in its entirety.

### TECHNICAL FIELD

The present disclosure is in the field of stable liposomes comprising entrapped amphipathic weak bases and an ammonium alkyl sulfonate.

### BACKGROUND

Liposomal compositions have been used successfully to deliver entrapped therapeutics. For example, Doxil® (Caelyx® in Europe) is a pegylated liposomal formulation including entrapped doxorubicin used for treatment of cancers such as ovarian cancer. Weak amphipathic bases like doxorubicin may be loaded into the liposomes using a transmembrane ion gradient. See, e.g., Nichols et al. (1976) Biochim. Biophys. Acta 455:269-271; Cramer et al (1977) Biochemical and Biophysical Research Communications 75(2):295-301). This loading method, generally referred to as remote loading, typically involves a drug having an ionizable amine group which is loaded by adding it to a suspension of liposomes prepared to have a lower inside/higher outside ion gradient, often a pH gradient. In addition, U.S. Patent Publication No. 20040219201 describes loading of weak amphipathic bases like doxorubicin driven by transmembrane gradient of ammonium glucuronate, which resulted in lack of intra-liposome doxorubicin crystallization and/or precipitation. However, such liposomes exhibit enhanced degradation upon long term 40° C. storage.

Gubernator (2011) Expert Opinion on Drug Delivery 8(5):565-580; describe remote loading of liposomes and Zhigaltsev et al. (2006) J. Control Release 110:378-86 describe the use of benzenesulfonate and hydroxybenzene sulfonate for drug precipitation (by complexation) inside the liposomes for improving drug retention. International patent application publication No. WO 93/00888 and South Korean patent application publication No. KR20030014780 describe the use of sulfonate, as counter ion for drug loading into liposomes.

Once the liposomes have drug loaded PLD (Pegylated Liposomal Doxorubicin) extravasated into interstitial tissues' fluids, little is known of the processes determining drug release. It is believed that gradual loss of the ammonium/proton gradients retaining the drug, enzymatic breakdown of liposomal phospholipids by phospholipases and/or endocytosis by scavenger macrophages likely contribute to drug release. Barenholz, (2012) J Control Release. 160(2):117-34. Doxorubicin when entrapped in the commercially-available liposomal Doxil® forms a salt with the divalent sulfate anion inside the liposome aqueous phase. In case of the bivalent sulfate as a counter ion, the doxorubicin-sulfate salt precipitate/aggregate in the intraliposome aqueous phase in the form crystal fibers (see, e.g., Haran et al (1993) Biochim Biophys Acta. 1151(2):201-15; Lasic et al (1992) FEBS Letts. 312(2-3):255-8. These crystals slow down further release rate from the effect of permeability coefficient which is determined mainly by liposome membrane composition and doxorubicin partition coefficient.

Although liposome-encapsulated doxorubicin is less cardio toxic than unencapsulated doxorubicin, preclinical and clinical data obtained from currently used pegylated liposomal formulations of doxorubicin confirm that there is very low release of drug from circulating liposomes (<5% of the injected dose). This allow the liposomes to reach the skin which is not a common target for liposomes and induce skin toxicity, namely the side effect palmar-plantar erythrodysesthesia (PPE), more commonly known as hand-foot syndrome. See, e.g., Gabizon et al (1994) Cancer Research 54:987-992; Solomon et al. (2008) Clinical Lymphoma and melanoma 1:21-32. PPE results in redness, tenderness, and peeling of the skin that can be uncomfortable and even painful. In clinical testing at 50 mg/m² dosing every 4 weeks, 50.6% of patients treated with Doxil® developed hand-foot syndrome. The prevalence of this side effect limits the Doxil® dose that can be given as compared with doxorubicin in the same treatment regimen.

The major factor which determines remote loading stability as well as kinetic order and rate of drug release from the liposome is the liposome lipid membrane composition (Zucker et al. (2009) J Control Release 139(1):73-80, Zucker et al. (2012) J Controlled Release, in press, Cohen et al (2012) J Controlled Release, in press). However, fine tuning of the release from transmembrane ion gradient driven remotely loaded liposomes can be achieved for example for ammonium sulfate driven loading by altering ammonium counter anion which affects the physical state of drug level and state of aggregation/gelation of precipitation of the amphipathic weak bases which are remote loaded by the transmembrane ammonium gradient (Wasserman et al (2007) Langmuir 23(4):1937-47; Zucker et al (2009), supra). In cases of remote loading of amphipathic weak bases, the type of the amphipathic weak base-counter ion will affect the level/state of active drug-counter ion salt precipitation and the level of drug intra-liposome precipitation has additional effect to this of liposome membrane composition. For any given amphipathic weak base the higher the precipitation the lower is the release rate (Wasserman et al (2007) Langmuir 23(4):1937-47). For example, while large increase in doxorubicin release rate due to change in liposome membrane composition will result in reduction in therapeutic index due to much lower drug level that will reach the tumor and higher drug level in unwanted tissues such as at the heart which may lead to reduction of therapeutic index. Drug release rate influences the pharmacokinetics, biodistribution, therapeutic activity, and toxicity of pegylated liposomal doxorubicin formulations in murine breast cancer. See, Charrois & Allen (2004) Biochim Biophys Acta. 1663(1-2):167-77.

Thus, there remains a need for chemically and physically stable liposomal formulations for delivering drugs, for example liposomes with reduced or eliminated intra-liposome crystallization/precipitation of entrapped weak amphipathic bases (drugs), for example to reduce unwanted side effects such as PPE without compromising the therapeutic efficacy.

### SUMMARY

The present invention relates to liposomes comprising an entrapped (i) amphipathic weak base and (ii) an alkyl-sulfonate salt or ion or aryl-sulfonate salt or ion. In one aspect, the invention relates to liposomes comprising an entrapped (i) amphipathic weak base and (ii) an alkyl sulfonate salt or ion. In other aspect, the invention relates to liposomes comprising an entrapped (i) amphipathic weak base and (ii) an aryl sulfonate salt or ion. In certain embodiments, the alkyl or aryl sulfonate salt or ion is an ammonium alkyl sulfonate or aryl sulfonate. In certain embodiments, the amphipathic weak base does not form crystals (non-amorphous higher order structures) within the liposomes, for example crystals of more than about 10 to 20 nm in diameter. In certain embodiments, the crystals are less than 20 nm in diameter. In other embodiments, the crystals are less than 20 nm in diameter. Any of the liposomes described herein may include some or no small amorphous precipitates. In other embodiments, at least 75% of the amphipathic weak base remains entrapped (and chemically stable) within the liposomes after at least 18 months at 4° C. or upon ten or more fold dilution at 37° C. for at least one week. In some embodiments, the liposomes are spherical in shape (rather than elliptical). In certain embodiments in which the liposome comprises an aryl sulfonate, magnesium is not present in the liposome. In any of the liposomes described herein, the alkyl sulfonate may be, for example, methanesulfonate, ethanesulfonate, 3-HydroxyPropane-1-Sulfonate, 2-HydroxyEthaneSulfonate, 1,3-Dihydroxy-2-(hydroxymethyl)-2-propanesulfinic acid, 2-Hydroxy-1-(2-hydroxyethoxy)-2-propanesulfonic acid, 4-Hydroxy-3,3-bis(hydroxymethyl)-1-butanesulfonic acid and the aryl sulfonate may be, for example, 4-HydroxyBenzene Sulfonate, 2,5-DihydroxyBenzeneSulfonate, 1,4-Dihydroxy-2-butanesulfonic acid, 2,3,4-Trihydroxybenzenesulfonic acid, 2,4,5-trihydroxybenzenesulfonic acid, 3,4-Dihydroxy-5-methoxybenzenesulfonic acid, or (3,4-Dihydroxyphenyl)(hydroxy)methanesulfonic acid. In certain embodiments, the logD value of the alkyl or aryl sulfonate counter-ion (at pH 5.5) is less than −3 (e.g., between −3 and −8), more preferably less than −4.5. Furthermore, any of the liposomes described herein may be pegylated.

The disclosure also provides compositions comprising these liposomes. In certain embodiments, the amphipathic weak base is doxorubicin, vincristine and/or one or more camptothecins such as topotecan. Also described are methods of making and using these liposomes for example by loading of amphipathic weak bases using a trans-membrane ammonium ion gradient having alkyl- or aryl-sulfonate as the ammonium to load an amphipathic weak base drug (e.g., doxorubicin, topotecan, etc.) into the liposomes. The alkyl or aryl sulfonate counter anions are distinguished from other monovalent counter ions in that they provide a high percentage (e.g., above 80-90%) stable drug loading while concomitantly retaining the chemical stability of the drug. The methods described herein also allow production of liposomes without change in the spherical shape of the liposomes from a sphere to an ellipse, where the change to the ellipsoid shape is indicative of the formation of crystals (non-amorphous structured molecules, typically larger than 10 nm in size) within the liposome when the ammonium counter ions is sulfate (e.g., doxorubicin-sulfate crystallization). This effect is suggested to contribute to the very long circulation time of doxorubicin administered as Doxil®.

Thus, in one aspect, described herein are liposomes comprising an amphipathic weak base and an alkyl or aryl sulfonate entrapped within the liposome. In certain embodiments, the alkyl or aryl sulfonate is an ammonium alkyl or aryl sulfonate. In certain embodiments, the amphipathic weak base is a chemotherapeutic agent, for example doxorubicin and/or topotecan. In certain embodiments, the liposomes are between about 20 to about 10000 nm in diameter. In other embodiments, the liposomes are between about 60 and 1000 nm in diameter. In certain embodiments, the liposomes comprise phospholipids, cholesterol and/or sphigolipids including ceramides (e.g., comprising any carbon chain from C2 to C22) and pegylated phospholipids in various ratios and concentrations, for example hydrogenated soy phosphatidyl choline (HSPC) in mole ratio of 45 to 70 and cholesterol in mole ratio of 30 to 50 and polyethyleneglycol(2000)-distearoyl-phospahtydil-ethanolamine (PEG-DSPE) in mole ratio of 2 to 20. In certain embodiments, the mole ratio of HSPC:cholesterol:PEG-DSPE is 54:41.5:4.5. In other embodiments, the liposomes comprise HSPC:PEG-DSPE:Ceramide in ratio 69.5:7.5:23. Any of the liposomes described herein may be formulated in a composition, for example, a composition comprising liposomes as described herein and further comprising one more pharmaceutically acceptable excipients or carriers. In certain embodiments, the composition comprises alkyl or aryl ammonium sulfonate.

In another aspect, methods of making liposomes as described herein provided. In certain embodiments, the liposomes are produced using an ammonium ion gradient, for example, where the amphipathic weak base is loaded into pre-formed liposomes against an ammonium ion gradient provided by an ammonium aryl or alkyl sulfonate (e.g., methanesulfonate) as a monovalent counterion. The gradient is capable of active transport of the weak amphipathic compound towards the inside of the liposomes (e.g., transport against the gradient). In any of the methods described herein, the concentration gradient of alkyl or aryl ammonium across the bilayer lipid membranes can be achieved by (i) preparing a suspension of liposomes, each liposome in the suspension having at least one internal aqueous compartment that contains a sulfonate derivative at a first (high) concentration, the liposomes suspended in an external bulk medium comprising the sulfonate derivative (e.g., ammonium methanesulfonate) at the first concentration; (ii) reducing (e.g., by dilution, dialysis, diafiltration and/or ion exchange) the first concentration of the sulfonate derivate in the external bulk medium (but not in internal aqueous compartment) to a lower, second concentration of the sulfonate derivative, thereby establishing an ammonium ion concentration gradient across lipid bilayers of the liposomes. In certain embodiments, sulfonate derivative is ammonium methanesulfonate. When the liposome suspension includes a weak amphipathic base, the base is transported to the inside of the liposomes by the gradient created after reducing the first concentration in the external medium. In certain embodiments making use of aryl sulfonate derivatives for loading, the preparation does not involve magnesium or calcium ions. In any of the methods described herein, preferably at least 50% of the amount of the weak amphipathic base (e.g., doxorubicin, topotecan) added to the suspension is transported to the inside of the liposomes. In another embodiment approximately 90% of the amount of the weak amphipathic base added to the suspension is transported to the inside of the liposomes. In specific embodiments, the loading efficiency for doxorubicin and for topotecan are greater than 90% and the weak amphipathic base to phospholipid ratio is in the range of about 10-3000 nmole/μmol respectively. Thus, also described are liposomes made by the methods described herein as well as compositions comprising the liposomes made by these methods.

In yet another aspect, uses of the liposomes and compositions comprising these liposomes as described herein, including use in methods of treating a condition susceptible to treatment using a composition comprising one or more liposomes prepared as described herein. In certain embodiments, the weak amphipathic base comprises a chemotherapeutic agent such as doxorubicin, vincristine and/or topotecan and the condition comprises a cancer. In other embodiments, the compositions further comprise the local anesthetics bupivacaine, and the condition comprises a cancer or pain management or many other applications in which an amphipathic weak base serve as the drug of choice. In any of these methods, additional (combination) therapies may also be used, concurrently or sequentially with the compositions described herein, for example additional chemotherapeutics. The liposomes and compositions comprising the liposomes as described herein can used in the manufacture of medicament for the treatment of any condition susceptible to treatment with liposomes comprising at least one weak amphipathic base.

In a still further aspect, methods of reducing the side effects associated with administration of liposomes with entrapped crystallized weak amphipathic bases (e.g., liposomal doxorubicin (Doxil®)), the methods comprising administering a liposomes (or a composition comprising the liposomes) as described herein to a subject in need thereof. In certain embodiments, the side effect comprises palmar-plantar erythrodysesthesia (PPE, also known as “hand and foot syndrome”).

These and other embodiments will readily occur to those of skill in the art in view of the disclosure herein.

### BRIEF DESCRIPTION OF THE FIGURES

FIG. 1, panels A to C, depict HPLC chromatograms at zero time (top column) and after incubation of four hours at 70° C. (bottom column) are presented for three anions. FIG. 1A shows chromatograms for doxorubicin in the presence of methanesulfonate at pH 5.80 and 500 mM concentration methanesulfonate. FIG. 1B shows chromatograms for doxorubicin in the presence of glucuronate at pH 6.03 and 250 mM concentration glucuronate. FIG. 1C shows chromatograms for doxorubicin in the presence of ammonium sulfate at pH 5.60 and 500 mM concentration ammonium sulfate. Each chromatogram shows two wavelength of detection, the upper is 254 nm and the lower 480 nm. The concentration loss of Doxorubicin in this short accelerated stability as calculated from the chromatograms is summarized in Table 3.

FIG. 2, panels A to G, depict cryo-transmission electron micrographs (CryoTEM) which compare commercial Doxil® and PLD of the same size and lipid composition which were remote loading using trans-membrane ammonium glucuronate and ammonium methanesulfonate. FIG. 2A shows Doxil® (Dox—Sulfate, scale bar: 100 nm). FIG. 2B shows liposomes loaded with doxorubicin in the presence of ammonium glucuronate (“DOXG,” scale bar: 50 nm). FIG. 2C shows liposomes loaded with doxorubicin in the presence of ammonium methanesulfonate (“DOXMS,” scale bar: 100 nm). FIG. 2D shows liposomes loaded with doxorubicin in the presence of ammonium ethanesulfonate. FIG. 2E shows liposomes loaded with doxorubicin in the presence of ammonium 4-hydroxybenzene sulfonate. FIG. 2F shows liposomes loaded with doxorubicin in the presence of ammonium 3-hydroxypropane sulfonate and FIG. 2G shows empty (lacking doxorubicin) liposomes ammonium methanesulfonate.

FIG. 3 is a graph depicting a PK comparison of blood levels of doxorubicin 48 hours after administration to mice of either Doxil® (“Doxil”) or ammonium methanesulfonate/doxorubicin liposomes (“DOX-046.2” or “DOXMS-050”). “DOX-046.2,” “DOXMS-050” and “DOXMS003” are all liposomes comprising ammonium methanesulfonate and differ only the concentration of ammonium methanesulfonate used to load the liposomes. “DOX046.2” was made using a 500 mM methanesulfonate gradient to load the doxorubicin into the liposomes; “DOXMS050” was made using a 350 mM methanesulfonate gradient and “DOXMS003” was made using a 250 mM methanesulfonate gradient. “DOX-046.2,” “DOXMS-050” and “DOXMS003” are also referred to as “PLDMS.”

FIG. 4, panels A and D, are graphs depicting a PK comparison as well as survival, body weight and average total scoring in rats following liposome administration. FIG. 4A shows a PK comparison of Doxil® and PLDMS (“DOX-046.003”) blood levels at 24 (left bar) and 48 hours (right bar) in mice. “DOX-046.003” (also referred to as “DOXMS003” was made using a 250 mM methanesulfonate gradient). FIG. 4B depicts survival of rats (according to humane end points) during as a function of the dose of drug administered. “DOXMS003” refers to ammonium methanesulfonate doxorubicin liposomes made using a 250 mM methanesulfonate gradient. The event was counted as death when the animal reached a humane end point as described previously. FIG. 4C is a graph depicting mean Body weight variations of the rats during the study for 1 mg/kg of Doxil® versus ammonium methanesulfonate doxorubicin liposomes (“DOXMS003”). FIG. 4D is a graph depicting average total scoring of rats during the study for 1 mg/kg of Doxil® versus ammonium methanesulfonate doxorubicin liposomes (“DOXMS003”).

FIG. 5, panels A to D, depict x-ray results of liposomal doxorubicin in presence of various ammonium sulfonate and sulfate salts. FIG. 5A shows results from (1)-DOX-MS (line “1” in the graph); DOX-SHPS (line “2” in the graph); DOX-4HBS (line “3” in the graph); DOX-ES (line “4” in the graph) and DOX-Sulfate (line “5” in the graph). FIG. 5B shows results using DOX-MS (left panel, labeled “Sample (1)”); DOX-4HBS (middle panel, labeled “Sample (3)”) and DOX-Sulfate (right panel, labeled “Sample (5)”). FIG. 5C shows scattered intensity of the indicated compositions. FIG. 5D shows scattered intensity of DOX-MS (labeled “(1)”); DOX-SHPS (labeled “(2)”); DOX-4HBS (labeled “(3)”); DOX-ES (labeled “(4)”); and DOX-Sulfate (labeled “(5)”).

FIG. 6 is a graph showing in vitro release profiles of liposomal doxorubicin in presence of various ammonium sulfonate salts (as indicated).

FIG. 7, panels A to F, are graphs presenting results of PLDMS/Doxorubicin/Saline comparison on mice body weight and tumor size.

FIG. 8 is a graph presenting results of a PK study PK003-LC100-120904 comparing healthy mice PK of PLDMS with Caelyx and free doxorubicin.

FIG. 9, panels A to D, are graphs presenting chemical results of doxorubicin in presence of various ammonium sulfonate (3HPS, 4HBS, ESA and MSA) and sulfate salts.

FIG. 10, panels A and B, are graphs showing healthy mice organ biodistribution of study PK003-LC100-120904 comparing healthy mice PK of PLDMS (middle bar) with Caelyx™ (left most bar) and free doxorubicin (right most bar, not present in T=48 hours).

### DETAILED DESCRIPTION

The practice of the present invention will employ, unless otherwise indicated, conventional methods of liposomology, physical chemistry, chemistry, biochemistry, physics, biophysics, pharmacy, and recombinant DNA techniques, within the skill of the art. Such techniques are explained fully in the literature. See, e.g., A. L. Lehninger, Biochemistry (Worth Publishers, Inc., current addition); Sambrook, et al. Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); and Methods In Enzymology (S. Colowick and N. Kaplan eds., Academic Press, Inc.). Lichtenberg and Barenholz 1988 LOP 134, Pharmaceutics 2nd edition Aulton M. E. ed. Churchill Livingstone, Harcourt Publishers 2002, London. New R.R.C. ed Liposomes Practical Approach 1^{st} edition, 1990. Torchilin V., et al eds: Liposomes Practical Approach 2^{nd} edition, Oxford Press 2003.

In describing the present invention, the following terms will be employed, and are intended to be defined as indicated below.

All publications, patents and patent applications cited herein, whether supra or infra, are hereby incorporated by reference in their entireties.

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a nucleic acid” includes a mixture of two or more such nucleic acids, and the like.

Before describing the compositions and methods in detail, it is to be understood that the disclosure is not limited to particular formulations or process parameters as such may, of course, vary. It is also to be understood that the terminology used herein is for the purpose of describing particular embodiments only, and is not intended to be limiting.

Although a number of methods and materials similar or equivalent to those described herein can be used, exemplary preferred materials and methods are described herein.

### Liposomes

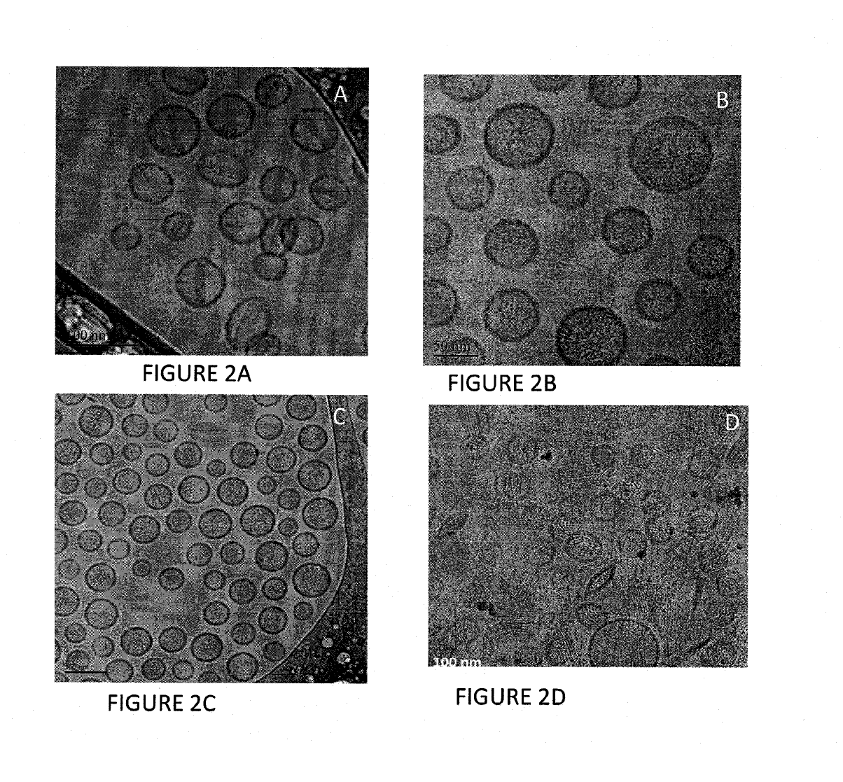

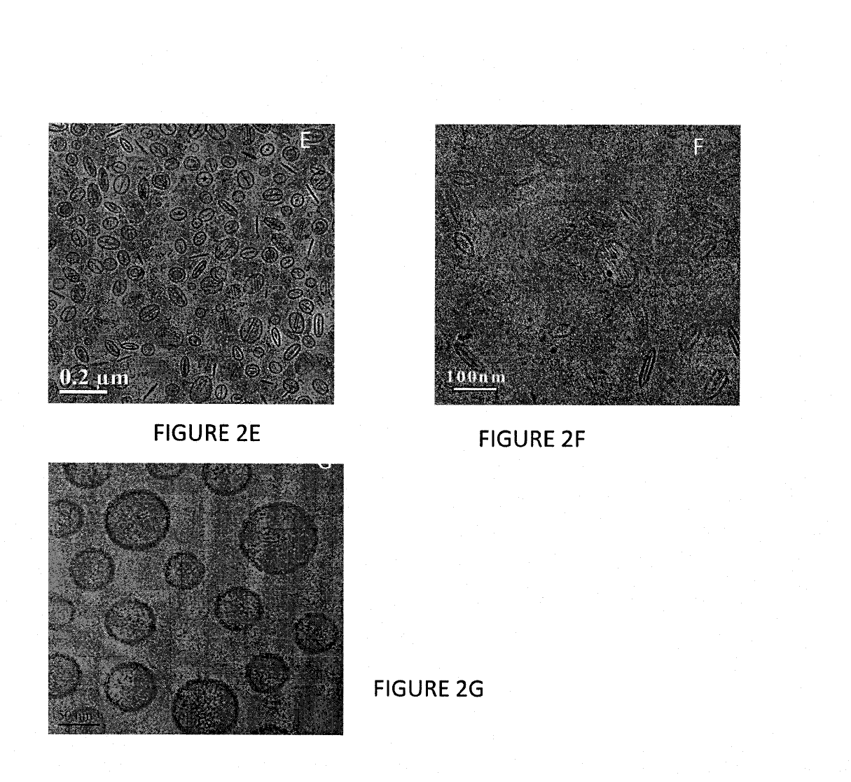

The present disclosure relates to liposomes where an amphipathic ionizable therapeutic agent (amphipathic weak base) is entrapped in the internal liposomal compartment(s) by an ammonium alkyl or aryl sulfonate. Entrapment is driven by a trans-membrane ammonium ion gradient and/or pH gradient. Thus, the liposomes comprise, in the intra-liposome aqueous phase, a salt of the amphipathic weak base with monovalent sulfonate anions. Some precipitates (e.g., small, amorphous particles) may be present within the liposome. However, larger, non-amorphous crystalline structures, particularly crystals above 10-20 nm, are not seen in the liposomes described herein. This is in contrast to Doxil®, for example, which includes one large crystal (typically the full-length of the entire liposome diameter, for example about 50 nm) in each liposome. The presence or absence of large, ordered crystal structures can be readily determined by X-ray and/or CryoTEM analyses (see, Examples) and/or by examining the shape of liposome. Liposomes that include higher molecular order crystals (such as Doxil®) are stretched into an elliptical (coffee bean) shape, whereas the claimed liposomes lacking such large crystals remain spherical. (See, FIG. 2).

Furthermore, the entrapped therapeutic agent retains at 37° C. a zero order slow release kinetics which is faster compared to the release rate of the agent entrapped in the liposomes in the form of an ionic salt with divalent sulfate anions, or with monovalent anion which is a derivative of aryl sulfonate. In particular, non-precipitated doxorubicin in presence of ammonium alkyl sulfonate within a liposomes exhibited a release percentage was 37-46% after three hours of incubation at 37° C. By contrast, remotely-loaded doxorubicin in presence of ammonium alkyl mono-valent sulfonate exhibited a release percentage of 25% and only 5% in presence of aryl sulfonates, following same incubation time (3 hours) at 37° C. See, Examples. Following five hours of incubation, non-precipitated doxorubicin in presence of ammonium alkyl sulfonate release percentage was <64% while following five hours of incubation, precipitated doxorubicin in presence of ammonium alkyl or aryl sulfonate release percentage was much lower, 44% for mono-valent alkyl and 24% for aryl. Furthermore, the release rate from the liposomes in the presence of mono-valent alkyl sulfonate (at 37° C.) is faster compared to the release rate of the agent entrapped in the liposomes in the form of an ionic salt with divalent sulfate anions, or with monovalent anion which is a derivative of aryl sulfonate. The liposomes described herein exhibit this faster release rate due to remote loading stability based on the monovalent alkyl and aryl ammonium sulfonate counter ions compared with a slower release rate achieved with the divalent sulfate as ammonium counter anions.

Liposomes suitable for use in the composition of the present invention include those composed primarily of vesicle-forming lipids. Vesicle-forming lipids, exemplified by the phospholipids, form spontaneously into bilayer vesicles in water. The liposomes can also include other lipids incorporated into the lipid bilayers, with the hydrophobic moiety in contact with the interior, hydrophobic region of the bilayer membrane, and the head group moiety oriented toward the exterior, polar surface of the bilayer membrane. See, e.g., Israelachvili (1980) Q. Rev Biophys. 13(2):121-200; Lichtenberg and Barenholz (1988) in Methods of Biochemcal Analysis, D. Glick (Ed), 33:337-462; Kumar (1991) Biophys J. 88:444-448. In preferred embodiments, the liposomes are spherical in shape as they do not contain one or more large crystals that tend to stretch the liposome into an elliptical shape.

Amphiphiles are defined by a packing parameter (PP), which is the ratio between the cross sectional areas of the hydrophobic and hydrophilic regions. Amphiphiles with a packing parameter of 0.74 to 1.0 (cylinder-like molecules) form a lamellar phase and have the potential to form liposomes. Amphiphiles with a larger packing parameter (inverted cone-shaped molecules) tend to form hexagonal type II (inverted hexagonal) phases. Such amphiphiles when having very small head group disperse hardly and in some cases do not even swell in the aqueous phase.

Amphiphiles with a smaller packing parameter of ≧⅔ (cone-shaped molecules) will self-aggregate as micelles. Examples of micelle forming amphiphiles which self-aggregate include phospholipids with short hydrocarbon chains, or lipids with long hydrocarbon chains (<10 carbon atoms), but with large, bulky polar head-groups (e.g. gangliosides and lipopolymers composed of a lipid to which a polyethylene glycol (PEG) moiety (≧750 Da) is covalently attached). See, e.g., Israelachvili (1992) in “Intermolecular and Surface Forces,” 2nd ed. Academic Press, pp 341-365; Lichtenberg and Barenholz, supra; Barenholz and Cevc (2000) in “Physical Chemistry of Biological Surfaces,” Marcel Dekker, NY, pp 171-241.

The vesicle-forming lipids are preferably ones having two hydrocarbon chains, typically acyl chains, and a head group, either polar or nonpolar. There are a variety of diacyl, dialkyl or one alkyl and one acyl chains, also one shingoid base and one acyl or alkyl chains. They can be synthetic, semi-synthetic, natural and natural originated but modified (such as partially and full hydrogenated soy PCs) vesicle-forming lipids, such as phospholipids, sphingomyelins, and some dialiphatic glycolipids, and glycosphingolipid which are defined as vesicle-forming lipids.

As defined herein, the term “phospholipids” includes but is not limited to phosphatidylcholine (PC), phosphatidic acid (PA), phosphatidylinositol (PI), phosphatidylserine (PS), sphingomyelin, PC plasmalogens, where the two hydrocarbon chains are typically between about 14-22 carbon atoms in length, and have varying degrees of unsaturation and having the two of the same hydrocarbon or two different hydrocarbons chains. The above-described lipids and phospholipids whose acyl chains have varying degrees of saturation can be obtained commercially or prepared according to published procedures.

The vesicle-forming lipid can be selected to have the gel to liquid crystalline [solid ordered to liquid disordered (SO to LD)] phase transition at the desired temperature range which allow achieving a specified degree of fluidity or rigidity, to control the stability of the liposome in serum and to control the rate of release of the entrapped agent in the liposome. Liposomes having a more rigid lipid bilayer, or a liquid crystalline bilayer, are achieved by incorporation of a relatively rigid lipid, e.g., a lipid having a relatively high SO to LD phase transition temperature range, e.g., above room temperature, more preferably above body temperature and up to 80° C. The SO to LD phase transition is also defined by Tm value, which is the temperature in which maximal change in the heat capacity during the phase transition occurs (Biltonen and Lichtenberg (1993) Chem. Phys. Lipids 64:129-142). Rigid, for instance saturated, lipids contribute to greater membrane rigidity in the lipid bilayer and concomitantly lower membrane permeability. Other lipid components, such as cholesterol and/or ceramides, are also known to contribute to membrane rigidity in lipid bilayer, High mole % cholesterol change the membrane lipid physical state to a Liquid Ordered (LO) phase Barenholz, Y. and Cevc, G., Structure and properties of membranes. In Physical Chemistry of Biological Surfaces (Baszkin, A. and Norde, W., eds.), Marcel Dekker, NY (2000) pp. 171-241.

For the sake of definition high fluidity is achieved by enriching the bilayer composed of a liposome forming with a large mole % of a lipid having its SO to LD phase transition at temperature range and Tm below 37° C. (namely below body temperature). On the contrary high rigidity (low fluidity) is achieved when the lipid bilayer is enriched with a liposome forming lipid having it SO to LD phase transition temperature range above the body temperature.

As a prerequisite in order to form liposomes, amphiphiles must be organized in a lamellar phase. However, the formation of lamellar phases is not sufficient to lead to liposome formation. See, Seddon (1990) Biochemistry 29(34):7997-8002. Liposome formation also requires the ability of the lamellae to close up on them to form vesicles. For example, some sphingolipids that form a lamellar phase are not able to form vesicles. See, e.g., Lichtenberg and Barenholz (1988), supra; Seddon (1990), supra; Barenholz and Cevc (2000), supra.

Other amphiphiles or lipids which are not liposome-forming lipids such as micelle forming lipids having packing parameter lower than 0.74 (such as lyso-PCs, Lyso-PGs, Lyso-Plas, lyso PIs, gangliosides, PEGylated lipids or detergents such as polysorbates (Tweens™) all having packing parameter below 0.74 (Garbuzenko et al (2005) Chem. Phys. Lipids 135:117-129) or lipids having their packing parameter larger than 1.0. Inverted phase forming lipids such as PEs, Cholesterol, and ceramides can also be added to the lipid bilayer as long as the additive packing parameter (APP), which is the summation of the packing parameters of all components multiply by the mole fraction of each of the forming the assembly is in the range of 0.74 to 1.0. See, e.g., Kumar et al. (1991) Proc Natl Acad Sci USA 88(2):444-448; Garbuzenko et al. (2005) Chem. Phys. Lipids 135:117-12; Khazanov et al. (2008) Langmuir 24:6965-6980.

The liposomes may optionally include a vesicle-forming lipid derivatized with a hydrophilic polymer (referred to as a lipopolymer), as has been described, for example in U.S. Pat. No. 5,013,556 and in WO 98/07409, which are hereby incorporated by reference in their entireties herein. As described above the lipopolymer comprises a micelle forming lipid having a packing parameter below 0.74. Other examples for pegylated lipids are include pegylated diglycerides (Ambegia et al. (2005) Biochimica et Biophysica Acta (BBA)—Biomembranes, 1669(2):155-163 and PEG-Ceramides (Zhigaltsev et al. (2006) J. of Controlled Release 110:378-386 (2006) and pegylated phosphatidic acid (Tirosh et al (1998) Biophys. J. 74, 1371-1379).

Addition (to the lipid mixture used for liposome preparation) of such a hydrophilic polymer-lipid conjugate at the mole fraction in which the APP of all lipids used for the liposomes is kept at the 0.74 to 1.0 range provides a liposome bilayer polymer provides a surface coating of hydrophilic polymer chains on both the inner and outer surfaces of the liposome lipid bilayer membranes. The outermost surface coating of hydrophilic polymer chains is effective to extend the blood circulation lifetime in vivo relative to liposomes lacking the polymer chain coating Gabizon et al (1994) Cancer Res. 54:987-992; Gabizon et al. (2003) Clin. Pharmacokinetics 42:419-436.

Similarly, liposomes having the lipopolymer present only in the external leaflet forming the liposome membrane can also be prepared by insertion of the lipopolymer such as PEGylated lipid to preformed liposomes and will have similar effect of prolongation of liposome circulation time. Lipids suitable for derivatization with a hydrophilic polymer include any of those lipids having a head group which allows covalent binding of the polymer listed above and, in particular PES, such as distearyl phosphatidylethanolamine (DSPE).

A preferred hydrophilic polymer chain is polyethyleneglycol (PEG), preferably as a PEG chain having a molecular weight between about 500 and about 15,000 Daltons, more preferably between about 750 and about 5,000 Daltons, most preferably between about 1,000 to about 3,000 Daltons. Methoxy or ethoxy-capped analogues of PEG are also preferred hydrophilic polymers, commercially available in a variety of polymer sizes, e.g., 120-20,000 Daltons. Preparation of vesicle-forming lipids derivatized with hydrophilic polymers has been described, for example in U.S. Pat. No. 5,395,619.

Preparation of liposomes including such derivatized lipids has also been described; where typically between 1-20 mole percent of such a derivatized lipid is included in the liposome formulation. It will be appreciated that the hydrophilic polymer may be stably covalently coupled to the lipid, or coupled through an unstable linkage which allows the coated liposomes to shed the coating of polymer chains as they circulate in the bloodstream or in response to a stimulus, as has been described, for example, in U.S. Pat. No. 6,043,094, which is incorporated by reference herein.

The liposomes described herein also include an entrapped alkyl or aryl sulfonate, preferably an ammonium alkyl or aryl sulfonate. The alkyl sulfonate may be, for example, methanesulfonate, ethanesulfonate, 3-HydroxyPropane-1-Sulfonate, 2-HydroxyEthaneSulfonate, 1,3-Dihydroxy-2-(hydroxymethyl)-2-propanesulfinic acid, 2-Hydroxy-1-(2-hydroxyethoxy)-2-propanesulfonic acid, 4-Hydroxy-3,3-bis(hydroxymethyl)-1-butanesulfonic acid and the aryl sulfonate may be, for example, 4-HydroxyBenzene Sulfonate, 2,5-DihydroxyBenzeneSulfonate, 1,4-Dihydroxy-2-butanesulfonic acid, 2,3,4-Trihydroxybenzenesulfonic acid, 2,4,5-trihydroxybenzenesulfonic acid, 3,4-Dihydroxy-5-methoxybenzenesulfonic acid, or (3,4-Dihydroxyphenyl)(hydroxy)methanesulfonic acid. In certain embodiments, the logD value of the alkyl or aryl sulfonate counter-ion (at pH 5.5) is less than −3 (e.g., between −3 and −8), more preferably less than −4.5.

The liposomes described herein may be formulated as pharmaceutical compositions, for example when admixed with an acceptable pharmaceutical diluent, carrier or excipient, such as a sterile aqueous solution, to give a range of final concentrations, depending on the intended use. The techniques of preparation are generally well known in the art as exemplified by Remington's Pharmaceutical Sciences, 16th Ed. Mack Publishing Company, 1980, incorporated herein by reference. For human administration, preparations should meet sterility, pyrogenicity, general safety and purity standards as required by FDA Office of Biological Standards.

### Liposome Preparation/Encapsulation

Also provided are methods for making liposomes as described herein, in certain embodiments, the method comprise a remote loading procedure for loading therapeutic agents (e.g., weak amphipathic bases) into pre-formed liposomes driven by an ammonium alkyl sulfonate gradient. The faster rate of release of the therapeutic agent from the liposomes made in this way affords flexibility to adjust dosing schedules without compromising the biological efficacy of the therapeutic agents. The instant disclosure therefore provides a beneficial alternative to loading by ammonium sulfate. The invention also provides extended shelf life product stability including doxorubicin and lipid chemical stability, doxorubicin encapsulation efficiency and encapsulation stability during storage.

Similar to the Doxil® trans membrane ammonium sulfate gradient driven drug loading method, the remote loading driven by trans membrane ammonium alkyl sulfonate gradient does not require the liposomes to be prepared in acidic pH, nor to alkalinize the extra-liposomal aqueous medium.

Previously-described liposomes loaded with lipophilic drugs using an ammonium aryl sulfonate resulted in liposomes including the lipophilic drug-alkyl sulfonate crystallized/precipitates (large, high molecular order structures within the liposome) in order to improve retention of the drug within the liposomes and release the drug more slowly from the liposome (see, e.g., Zhigaltsev et al. (2006) Journal of Controlled Release 110:378-386). By contrast, the liposomes described herein include an amphipathic (not lipophilic) drug (e.g., doxorubicin) and, in addition, include little or no crystallized (or precipitated) drug. In addition, the logD of the alkyl or aryl sulfonate counter-ion (at pH 5.5) is less than −3 (e.g., between −3 and −8), more preferably less than −4.5. Furthermore, liposomes as described herein made with aryl sulfonates use a trans membrane ammonium gradient for remote loading and do not require the use of a proton gradient, the proton gradient achieved in Zhigaltsev et al. (2006) by using a either magnesium sulfate gradient or a calcium hydroxybenzenesulfonate gradient formed by dialyzing the LUVs against HEPES-buffered sucrose solutions (pH 6.5) and subsequent addition of the ionophore A23187 to the suspension of the LUVs resulting in the outward movement of one metal cation in exchange for two protons, thus establishing a transmembrane pH gradient, which drives drug uptake. Thus, the liposomes prepared using an ammonium aryl sulfonate gradient as described herein may not comprise magnesium or calcium ions and are not necessarily made using magnesium and or calcium ions.

In addition, unlike previously-described liposomes including a weak amphipathic drug such as doxorubicin (e.g., Doxil®), the weak amphipathic drug entrapped within the liposomes described herein does not form crystals (e.g., crystals of larger than 10 nm in diameter) within the liposome, resulting in liposomes of elliptical shape. In the absence of the formation of these relatively large crystals, the liposomes retain their spherical shape (FIG. 2) and, in addition, show significant differences from Doxil® in terms of release rate and reduced adverse effects when administered to a patient. See, Examples.

The approach described herein that makes use of loading driven by ammonium alkyl or aryl sulfonate salts also permits the loading of therapeutic agents in a broad spectrum of liposomes of various types, sizes, and compositions, including sterically-stabilized liposomes, immunoliposomes and sterically-stabilized immunoliposomes. “Encapsulated” as used herein refers to an agent entrapped within the aqueous spaces of the liposomes or within the lipid bilayer.

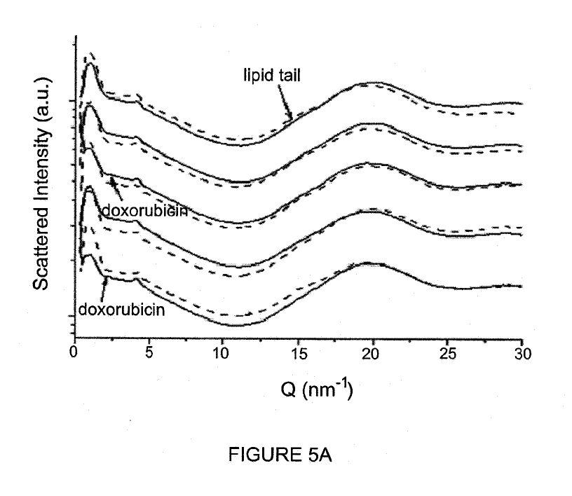

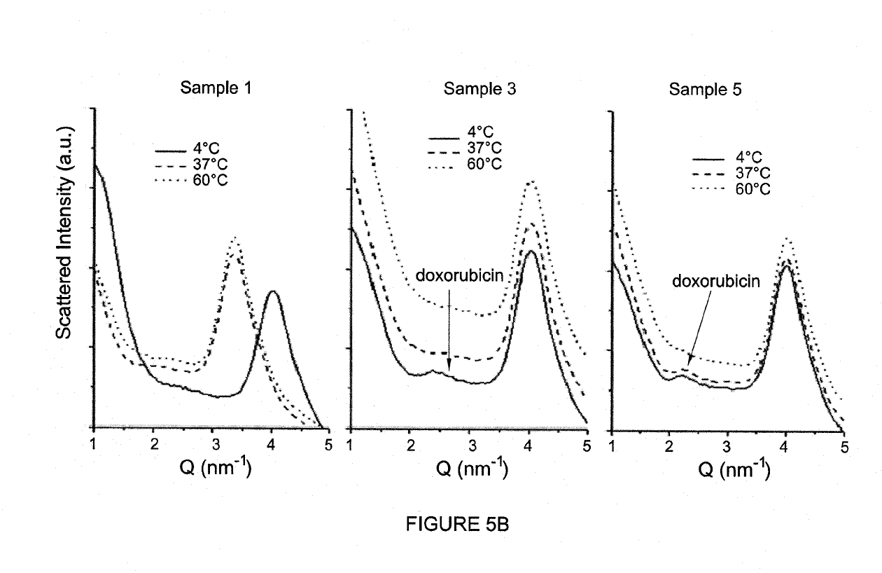

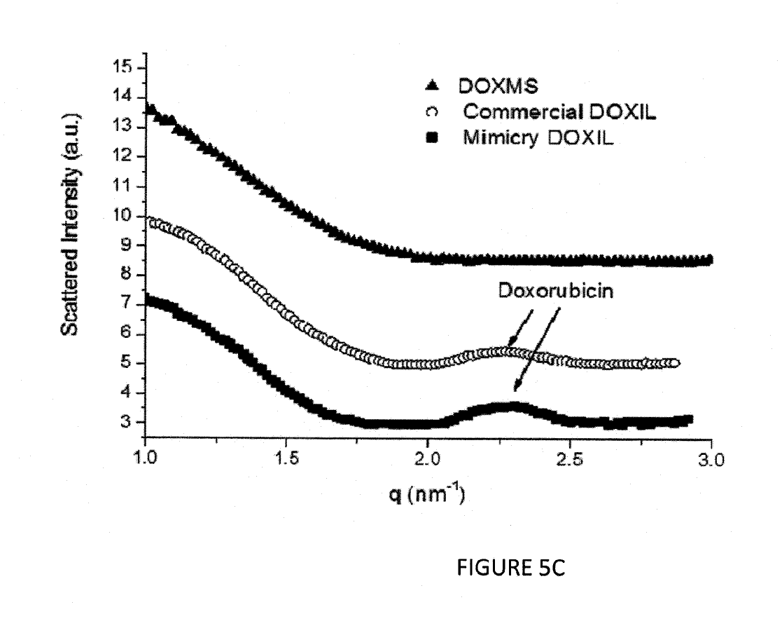

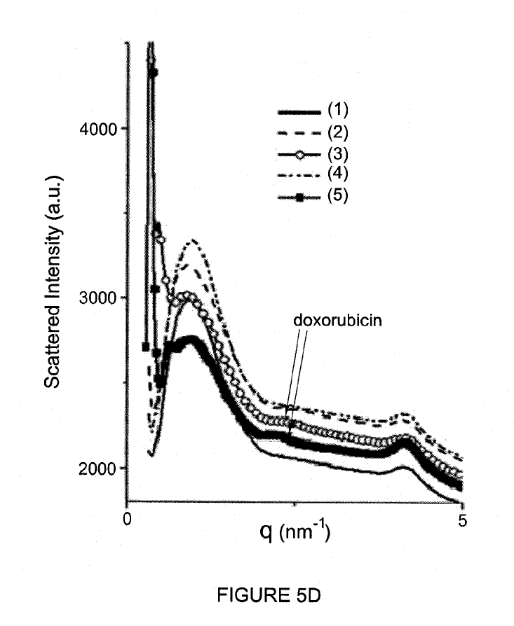

The increased release rate of the encapsulated compound is a result of using alkyl or aryl sulfonate as the balancing (counter) anion. While not wishing to be bound by one theory, it is hypothesized that the alkyl or aryl sulfonate ion, being monovalent, is less effective compared to a sulfate ion at inducing aggregation and precipitation of the therapeutic agent after being transported inside the liposomes. The inventors have observed that the solubility of doxorubicin is approximately 30-fold greater (or more) in a 250 mM ammonium alkyl or aryl sulfonate solution than in a 250 mM ammonium sulfate solution as determined by comparing ammonium alkyl or aryl sulfonate to ammonium sulfate. In addition, doxorubicin precipitates at less than 2 mM concentration in the presence of sulfate ions, while doxorubicin solubility in ammonium alkyl or aryl sulfonate is similar to the maximal water solubility of doxorubicin HCl (50 mg/ml, see, Sigma catalog) without precipitating. Doxorubicin HCl at 70 mM did not precipitate in the presence of alkyl or aryl sulfonate ions while in ammonium sulfate precipitation occurs at less than 2 mM (namely at least 35 fold higher solubility of the methanesulfonate form). Accordingly, when alkyl sulfonate is the counter anion, most of the therapeutic agent is in a soluble form and therefore it is more available for release from the liposomes. Thus, whereas aryl and alkyl sulfonate liposomes as described herein do not include one or more large crystals (e.g., 10 nm or more) at 37° C., sulfonate precipitation was observed even at 37° C. (see, e.g., FIG. 5B).

Furthermore, the permeability of alkyl or aryl sulfonate through lipid membranes can be predicted from logP values (see, e.g., Stein D. 1986, Transport and diffusion across cell membranes, Chapter 2. Academic Press, Orlando, Fla.) and/or logD values. Log P (and more specifically, logD at pH=5.5) values which described in Table 1A and 1B suggest that permeability of alkyl or aryl sulfonate through the liposomal membranes is very low and similar to both glucuronate (<−2.3) and sulfate ions. The low LogP and LogD values (which determine permeability Coefficients (Stein W. D. et al. 1986)) and provide for alkyl or aryl sulfonate ion gradients for loading of the amphipathic weak bases. In certain embodiments, the LogD values (at pH 5.5) are below about −3 (e.g., between about −3 and −8) and even more preferably less than about −4.5.

The method of the current invention can be used to remotely load essentially any therapeutic agent which is amphipathic weak base which being proton-able it can exist in a positively charged state, or in charge less state dependent on aqueous medium pH. Preferably, the agent should be amphipathic so that it will partition into the lipid vesicle membranes. Also, preferably, the therapeutic compound suitable for loading is a weak amphipathic base compound.

Liposomal suspensions comprised of liposomes having an ion gradient across the liposome bilayer (also referred to as “a trans-membrane ion” and/or “pH gradient”) for use in remote loading can be prepared by a variety of techniques, such as those detailed in Szoka et al. (1980) Ann Rev Biophys Bioeng 9:467 and Lichtenberg and Barenholz (1988) in “Methods of Biochemical Analysis” (Glick, D., ed.) Wiley, NY, 33, pp. 337-462.

Multi-lamellar vesicles (MLVs) can be formed by simple lipid-film hydration techniques. In this procedure, a mixture of liposome-forming lipids (see above) with and without other lipids of the type described above is dissolved in a suitable organic solvent and the solvent is later evaporated off or lyophilized leaving behind a thin film or a dried powder “cake” (respectively). The film or dry cake is then hydrated by the desired aqueous medium, containing the solute species, e.g., ammonium alkyl or aryl sulfonate, which forms the aqueous phase in the liposome interior volume and also the extra-liposomal suspending solution. The lipid film is hydrates to form LVs, typically with sizes between about 0.1 to 10 microns.

The lipids used in forming the liposomes of the present invention are preferably present in a mole % of about 50-100 mole percent vesicle-forming lipids, with or without cholesterol and optionally 1-20 mole percent of a lipid derivatized with a hydrophilic polymer chain such as PEG. One exemplary formulation includes 80-90 mole percent phosphatidylcholine, 1-20 mole percent of PEG-DSPE. Cholesterol may be included in the formulation at between about 1-50 mole %. In a preferred embodiment, the lipid components are hydrogenated soy phosphatidylcholine (HSPC), cholesterol (Chol) and mono methoxy-capped polyethylene glycol of 2000 Da derivatized distearoyl phosphatidylethanolamine abbreviated as (mPEG (2000)-DSPE, or PEG-DSPE) in a mole % of between about 50 and 60 (HPSC), 35-50 (cholesterol) and 4-10 mole % (PEG-DSPE), for example of the mole ratio of 54.5:41:4.5. for the 3 above components respectively.

For preparation liposomes comprising ammonium alkyl or aryl sulfonate using a trans membrane gradient, the lipid hydration medium contains ammonium alkyl or aryl sulfonate. It will be apparent that the concentration of ammonium alkyl or aryl sulfonate depends on the amount of therapeutic agent to be loaded. Typically, the concentration is between 50 to 750 mM of alkyl or aryl sulfonate as ammonium salt. In preferred embodiments, the hydration medium contains 250 mM, 350 mM or 500 mM alkyl or aryl sulfonate as ammonium salt.

The vesicles formed by the thin film or dry cake mechanical dispersion method may be sized to achieve a size distribution within a selected range, according to known methods. Small unilamellar vesicles (SUVs) defined as liposomes in the range 20 to 100 nm diameters at a narrow size distribution in this range can be prepared by post-formation ultrasonic irradiation, or homogenization, or extrusion. Homogeneously sized liposomes having sizes in a selected range between about 50 nm to 400 nm can be produced, e.g., by extrusion through polycarbonate membranes or other defined pore size membranes having selected uniform pore sizes ranging from 30 to 1000 nm, for example, 50, 80, 100, 200 or 400 nm. The pore size of the membrane corresponds roughly to the largest size of liposomes produced by extrusion through that membrane, particularly where the preparation is extruded two or more times through the same membrane. The sizing is preferably carried out in the original lipid-hydrating buffer, so that the liposome interior spaces retain this medium as an intraliposome aqueous phase throughout the sizing processing steps. In the case of remotely loaded therapeutics, the therapeutic agent is loaded into the preformed liposomes after their sizing. Remote loading is different from passive loading for the latter the drug is present in the hydration medium and therefore it is encapsulated during the stage of hydration.

A “remote” or “active” loading process requires firstly creation of an ion (i.e. ammonium ion) gradient by exhaustive dialysis or equivalent approaches such as exhaustive diafiltration, or gel exclusion chromatography (Haran et al. (1993) Biochim. Biophys. Acta 1151:201-215 and U.S. Pat. Nos. 5,192,549 and 5,244,574, incorporated in their entireties herein. For example, for small-scale preparation, the gradient can be created by four consecutive dialysis exchanges against at least 50 volumes of the dialysis buffer. For large-scale preparation, the gradient may be prepared by a three-step tangential flow dialysis, e.g., using a Minitan ultrafiltration system (Millipore Corp., Bedford, Mass.) equipped with“300 K” polysulfone membranes. The dialysis buffer contains electrolytes (e.g., sodium chloride or potassium chloride) or non-electrolytes (glucose or sucrose). In one preferred embodiment, the dialysis buffer is 15 mM HEPES containing 5% dextrose at approximately pH 7. Using either of the dialysis approaches (large or small-scale) and under conditions in which the hydration medium was 60-500 mM ammonium alkyl or aryl sulfonate salt, a gradient of 1,000 or higher can be obtained without dilution of the liposomal dispersion.

The mechanism of remote loading driven by an ammonium ion trans-membrane gradient is described in Haran et al 1993, supra; U.S. Pat. Nos. 5,192,549 and 5,244,574 and Zucker et al (2009) J. Controlled Release 139:73-80. In brief, the trans membrane ammonium gradient leading to small amount of the neutral ammonia gas present in the intra-liposomal aqueous phase to be released fast of the liposomes as due to its high permeability coefficient of around 1.3×10⁻¹ cm/second. The efflux of ammonia shifts the equilibrium within the liposome toward production of excess of protons which results in a [H+] gradient (lowering the intraliposome pH), with the intraliposomal concentration higher than that in the extraliposomal medium The low pH stops the formation of neutral ammonia gas. In addition in the intra-liposome aqueous phase an excess of alkyl or aryl sulfonate ions over the ammonium ion is created. Unprotonated un-charged drug present in the external liposome medium diffuses across the liposomal lipid bilayer into the intra-liposome aqueous phase were it becomes protonated and charged so it can bind the excess of the counter anion of the ammonium (e.g., alkyl or aryl sulfonate) present in the intra-liposome aqueous phase.

Thus, the remote loading results from exchange of the therapeutic agent added to the external or bulk medium in which the preformed sized-liposomes are suspended with the ammonium ions present in the internal liposomal aqueous phase (Haran et al. (1993), supra). The efficiency of loading depends, to large extent, on the ammonium ion gradient, where before the remote loading the concentration of the ammonium ion inside the liposomes is much higher than the concentration of ammonium ion in the external, liposomes' medium. The magnitude of this gradient determines to a large extent the level of encapsulation; the larger the gradient and the higher is the internal ammonium ion concentration, generally the higher the encapsulation. See, e.g., Clerc and Barenholz (1998) Anal. Biochem. 259:104-111; Zucker et al (2009) J. Controlled Release 139:73-80.

An ammonium alkyl or aryl sulfonate trans membrane gradient, where the ammonium ion concentration is much higher in the intra-liposome aqueous phase than in the external liposome suspension medium (i.e., a higher inside/lower outside ammonium ion gradient) may be formed in a variety of ways, for instant, by (i) controlled dilution of the external medium, (ii) dialysis against the desired final medium, (iii) molecular-sieve gel permeation chromatography, e.g., using Sephadex G-50, and elution medium lacking ammonium ions, or (iv) high-speed centrifugation and re-suspension of pelleted liposomes in the desired final medium (Haran et al. (1993), supra). The final external medium selected will depend on the mechanism of gradient formation and the external ion concentration desired.

The gradient is measured by measuring ammonium in the external liposome medium and the intraliposome ammonium concentration by ammonium or ammonia electrodes (Haran et al. (1993), supra) as the ratio of ammonium alkyl or aryl sulfonate inside to that outside of the liposomes. Generally, the gradient (the above ratio) is in the range of 10 to 1000 inside/outside. Preferably, the gradient is in the range of 100-10000.

The concentration of ammonium alkyl or aryl sulfonate in an external medium that also contains electrolytes may be measured as ammonia concentration at pH 13-14 (see, Bolotin et al. (1994) J. Liposome Research 4(i):455-479) by an ion analyzer, e.g., a Corning 250 pH/ion analyzer (Corning Science Products, Corning, N.Y.) equipped with a Corning 476130 ammonia electrode and an automatic temperature compensation (ATC) stainless steel probe. A proton gradient across the lipid bilayer of the liposomes is produced in parallel as a result of creation of the trans-membrane ammonium gradient (Haran et al. (1993), supra; U.S. Pat. No. 5,192,549). Optionally, the external medium is exchanged by a medium lacking ammonium alkyl or aryl sulfonate salt, for example it is replaced by a salt such as NaCl or KCl, or by a sugar such as dextrose or sucrose that gives similar osmolality inside and outside of the liposomes, or osmolality that does not affect liposome physical stability.

The remote loading is preferably carried out at a temperature above the phase transition temperature of the liposome forming lipids. Thus, for liposomes formed predominantly of saturated phospholipids such as DPPC, DSPC or HSPC, or N-palmitoyl sphnogomylin the loading temperature may be as high as 60° C. or even higher. The loading duration is typically between 15-120 minutes, depending on rate of permeability of the drug to via the liposome bilayer membrane, the temperature, and the relative concentrations of liposome lipid and drug. In one preferred embodiment, the loading is performed at 60° C. and for 60 minutes (for more details see Haran et al. (1993), supra; Zucker et al (2009), supra).

Thus, with proper selection of liposome concentration, external concentration of added compound, and the ion gradient, essentially all of the added compound may be loaded into the liposomes. For example, with an trans membrane ammonium ion gradient of approximately 1000, encapsulation of doxorubicin can be greater than 90% and even >95%. Knowing the calculated internal liposome volume, and the maximum concentration of loaded drug, one can then select an amount of drug in the external medium which leads to substantially complete loading into the liposomes.

If drug loading is not effective to substantially deplete the external medium of free drug, the liposome suspension may be treated, following drug loading, to remove non-encapsulated drug. Free drug can be removed, for example, by ion exchange chromatography, molecular sieve chromatography, dialysis, or centrifugation. In one embodiment, the non-entrapped drug is removed using the cation exchanger Dowex 50WX-4 (Dow Chemical, MI). For example, free doxorubicin binds to a cation exchange resin while liposomal doxorubicin when encapsulated in neutral or negatively charged liposomes is not binding to this cation exchanger (Storm et al. (1985) Biochim Biophys Acta 818:343; Amselem et al (1990) J. Pharm. Sci. 79:1045-1052).

### Amphipathic Drugs

Any amphipathic weak base drug can be entrapped within a liposome with ammonium methanesulfonate as described herein. Examples of therapeutic agents which can be loaded into liposomes by the method of the invention include, but are not limited to, doxorubicin, mitomycin, bleomycin, daunorubicin, streptozocin, vinblastine, vincristine, mechlorethamine hydrochloride, melphalan, cyclophosphamide, triethylenethiophosphoramide, carmustine, lomustine, semustine, fluoruracil, hydroxyurea, thioguanine, cytarabine, floxuridine, decarbazine, cisplatin, procarbazine, ciprofloxacin, epirubicin, carcinomycin, N-acetyladriamycin, rubidazone, 5-imidodaunomycin, N-acetyldaunomycine, all anthracyline drugs, daunoryline, propranolol, pentamindine, dibucaine, tetracaine, procaine, chlorpromazine, pilocarpine, physostigmine, neostigmine, chloroquine, amodiaquine, chloroguanide, primaquine, mefloquine, quinine, pridinol, prodipine, benztropine mesylate, trihexyphenidyl hydrochloride, propranolol, timolol, pindolol, quinacrine, benadryl, promethazine, dopamine, serotonin, epinephrine, codeine, meperidine, methadone, morphine, atropine, decyclomine, methixene, propantheline, imipramine, amitriptyline, doxepin, desipramine, quinidine, propranolol, lidocaine, chlorpromazine, promethazine, perphenazine, acridine orange, prostaglandins, and other molecules similar to these above.

In certain embodiments, the weak amphipathic base is doxorubicin, topotecan and the like. Doxorubicin loaded in liposomes (e.g., liposomes having an external surface coating of hydrophilic polymer [poly ethylene glycol (PEG) chains]) by a trans membrane ammonium alkyl or aryl sulfonate gradient (methanesulfonate also referred to herein as “PLD-MS”) remain spherical following drug loading and exhibit a relative faster release rate than currently used doxorubicin liposomes (Doxil®/Caelyx® also referred to as PLD) while exhibiting similar therapeutic efficacy against tumors (see Examples).

### Administration

The liposomes and compositions comprising these liposomes as described herein can be administered by any suitable method, including, but not limited to, intravenous, intramuscular, oral, intraperitoneal, intraocular, subcutaneous routes of administration.

The liposomes and compositions comprising these liposomes described herein can be administered alone (in one or more doses) or as part of a combination therapy, for example with other chemotherapeutic agents (e.g., liposomes or other therapeutics). While specific time intervals and courses of treatment will vary depending on the extent of symptoms and the condition of the patient.

### Applications

The liposomes and compositions comprising these liposomes as described herein comprise an amphipathic weak base (drug) and ammonium alkyl or aryl-sulfonate. These liposomes do not contain large crystals within their internal compartment (e.g., crystals larger than 10-20 nm in size) and are typically spherical in shape. In addition, the liposomes load at least 80% of the drug (e.g., at least 80%, more preferably at least 90% and even more preferably at least 95% stable drug loading) and, in addition, the drug maintains its chemical stability within the liposome. Thus, the liposomes described herein enhance treatment and/or prevention of any of the diseases or conditions treated by the entrapped drug. In certain embodiments, the drug is a chemotherapeutic agent such as doxorubicin and the disease is a cancer (e.g., ovarian, breast, etc.).

Furthermore, the compositions described herein exhibit relatively faster release rates of the entrapped drug in vivo (e.g., as compared to other liposomal formulations such as Doxil®). Therefore, the liposomes described herein reduce the side effects associated with the entrapped drug, as the opportunity for the drug to accumulate in non-targeted tissues (for example, skin when targeting a tumor) is reduced and side effects such as palmar-plantar erythrodysesthesia (PPE), and mucositis or asthenia, sleep disruptions and alimentary tract organs side effects observed in patients and animals treated with liposomal chemotherapeutics (such as Doxil®) are reduced. See, e.g., Hackbarth et al. (2008) Support Care Cancer 16(3):267-73.

### EXAMPLES

Below are examples of specific embodiments for carrying out the present disclosure. The examples are offered for illustrative purposes only, and are not intended to limit the scope of the present disclosure in any way.

### Example 1

### Counter-Ion Screening

The screening process for the most suitable counter ion for generation of stable liposome compositions in which the entrapped amphipathic base remains chemically stable included the following steps. First, the relevant physicochemical properties of a large group of ammonium counter ions were compared at pH 5.5 to 6.0 and the ability of these counter ions to induce a precipitation of doxorubicin was studied.

### A. Doxorubicin

As shown in Table 1, only monovalent (at the specified pH of the experiment) did not induce doxorubicin precipitation while the bivalent and trivalent counter ions used (at this pH) induce precipitation. All logP and logD data from the chemspider website on the internet.

TABLE 1
physicochemical properties and the effect of counter anion on doxorubicin precipitation

| | | LogD | | | | Experimental | |
|---|---|---|---|---|---|---|---|
| Ammonium anions at | LogP | (acid)- | Acid | Acid | Acid | pH of the | |
| measured pH | (acid) | at pH = 5.5 | pKa1 | pKa2 | pKa3 | precipitation | Precipitation |
| Sulfate (SO4)⁻² | −1.114 | −5.61 | −3 | 1.96 | | 5.5 | Yes |
| Citrate (C6H6O7)⁻² | −1.198 | −6 | 3.15 | 4.77 | 6.4 | 5.2 | Yes |
| Pyruvate (C3H3O3)⁻ | −1.24 | −4.04 | 2.49 | | | 5.2 | No |
| Lactate (C3H5O3)⁻ | −0.85 | −2.82 | 3.86 | | | 6.2 | No |
| Maleate (C4H2O4)⁻ | −0.008 | −3.41 | 1.92 | 6.27 | | 5.2 | No |
| Gluconate (C6H11O7)⁻ | −2.116 | −4.83 | 3.86 | | | 5.2 | No |
| Glycolate (C2H3O3)⁻ | −1.204 | −3.38 | 3.83 | | | 5.2 | No |
| Tartarate (C4H5O6)⁻² | −1.081 | −5.52 | 2.98 | 4.34 | | 4.5, 5.0 | Yes |
| Borate (H2BO4)⁻ | −0.61 | −0.61 | 9.24 | | | 5.5, 6.0 | No |
| Methanesulfonate | −2.087 | −5.39 | −2 | | | 5, 6 | No |
| (CH3O3S)⁻ | | | | | | | |
| Glucuronate | −2.57 | −5.38 | 3.18 | | | 5.2 | No |
| (C6H9O7)⁻ | | | | | | | |
| Ethanesulfonate | −1.577 | −4.85 | ≈−2 | | | 5.5 | No |
| (C₂H5O3S)⁻ | | | | | | | |
| 3-HydroxyPropane-1- | −2.59 | −5.39 | ≈−2 | | | 5.5 | No |
| Sulfonate (C₃H₇O₄S)⁻ | | | | | | | |
| 4-HydroxyBenzene | −1.624 | −5.12 | ≈−2 | | | 5.5 | No |
| Sulfonate (C₆H₅O₄S)⁻ | | | | | | | |
| ** 2-HydroxyEthaneSul- | −2.815 | −6.25 | ≈−2 | | | | |
| fonate (C2H₅O₄S)⁻* | | | | | | | |
| ** 2,5-DihydroxyBenzeneSul- | −2.284 | −5.78 | ≈−2 | | | | |
| fonate (C₁₀H₁₆NO₅S)⁻ | | | | | | | |

** theoretical anions

Mono-valent ammonium sulfonate salts having logD lower than about (−4.5) at pH=5.5, will probably encapsulate efficiency doxorubicin into liposomes for a long time.

Only ammonium counter anions that did not induce doxorubicin precipitation (monovalent ions) and exhibited a low enough Log D at pH 5.5 and therefore membrane permeability coefficient which can be calculated and predicted from Log P and Log D values (Stein W. D., 1986 Transport and Diffusion across membranes, Academic Press NY Chapter 1) were tested for accelerated chemical stability test (4 hours, 70° C.).

For accelerated stability the, chemical stability of doxorubicin in various ammonium-anions salts solutions was tested. Briefly, doxorubicin (2 mg/ml) was dissolved in various ammonium-anions salt solutions and incubated for 4 hours at the temperature of 70° C. At the end of 4 hours incubation doxorubicin concentration and the presence of doxorubicin degradation products were analyzed by HPLC using Agilent 1100 according to following protocol: Mobile phase: 50% MeOH, 40% phosphate buffer, pH=4, 10% Acetonitrile and 2% TEA Column: Phenomenex, Luna, C-18, Flow: 1 ml/min, detection wavelength 254 and 480 nm.

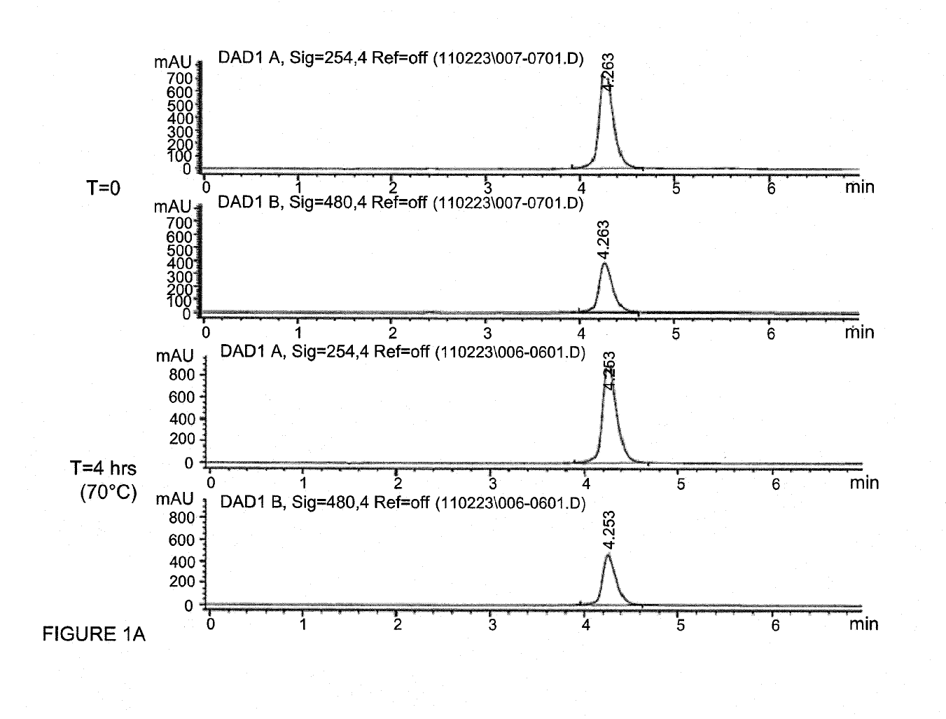

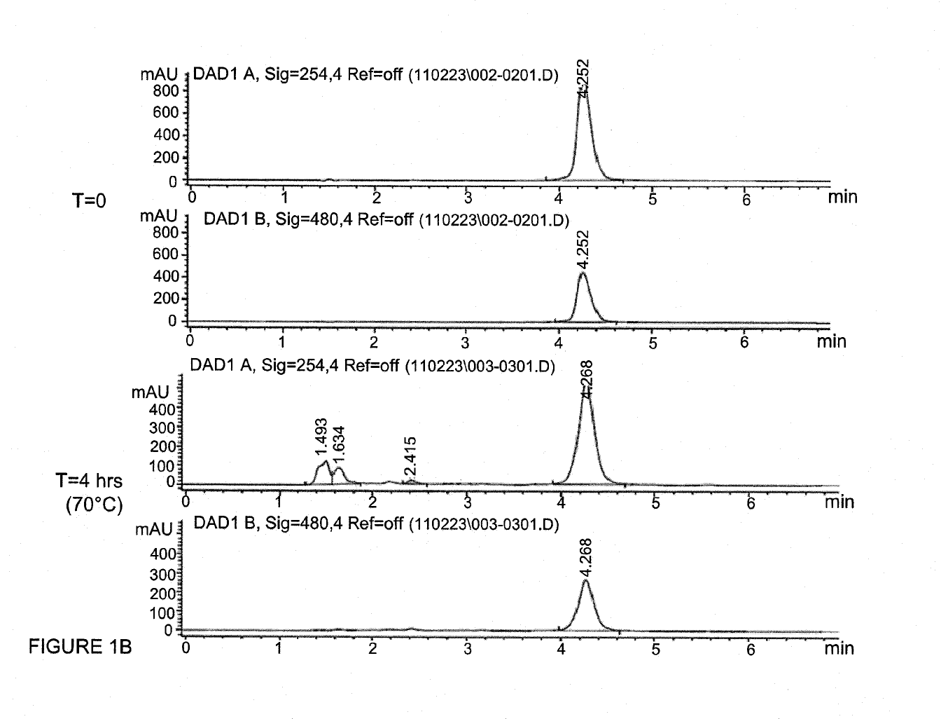

The results are shown in FIG. 1 and Table 2.

TABLE 2
Doxorubicin stability

| Ammonium | Conc. | pH after | Doxorubicin |
|---|---|---|---|
| anions | (mM) | titration | chemical |
| Pyruvate | 500 | 5.2, 6 | Stable |
| Lactate | 250, 500 | 3.5, 4, | Stable |
| | | 4.5, 5.5, 6 | |
| Maleate | 500 | 4.5, 5.2 | Stable |
| Gluconate | 500 | 5.2, 6 | Not stable |
| Glycolate | 500 | 5, 6 | Stable |
| Borate | 500 | 5.5, 6 | Not stable |
| Methanesulfonate | 250, 350, | 5, 6 | Stable |
| | 500 | | |
| Glucuronate | 250 | 3, 3.5, | Not stable |
| | | 4, 4.5 | |
| 3-HydroxyPropane-1- | 500 | 5.5 | Stable |
| 4-HydroxyBenzene | 500 | 5.5 | Stable |
| Sulfonate | | | |
| Ethanesulfonate | 500 | 5.5 | Stable |

In addition, the concentration loss of Doxorubicin in this short accelerated stability as calculated from the chromatograms is summarized in Table 3 (and shown in FIG. 1).

TABLE 3
Doxorubicin accelerated chemical stability in various ammonium solutions

| ammonium ion | Assay loss (%) at 254 nm | Additional peaks |
|---|---|---|
| ammonium | <15 | no |
| methanesulfonate | | |
| ammonium sulfate | <15 | no |
| ammonium glucuronate | 34 | yes |

All ammonium anions that passed successfully both the precipitation test (clear solution in the presence of doxorubicin) and accelerated chemical stability of Doxorubicin continued to the next screening stage in which the stability of doxorubicin in remotely loaded liposomes differing in their ammonium salts (that show good results with the 2 first screening tests) were compared.

### B. Topotecan

For details on suitability of topotecan remote loading by transmembrane ammonium gradient loading are described by Zucker et al, JCR, 2009, 139, 73-8. in this example we compared remoter loading of topotecan using sulfate and methanesulfonate and ammonium counter ion.

Remote loading of topotecan were performed in the same way as the experiments described in Table 4 and Table 5 below for doxorubicin remote loading except that the drug concentration was determined from its absorbance at a wave length of 370 nm (which is the wavelength of maximum absorbance of topotecan).

The experiment demonstrates that topotecan (as HCl salt), 1.3 mg/ml (2.84 mM), encapsulation efficiency into liposomes exhibiting trans membrane gradient of ammonium (350 mM) and having methanesulfonate (350 mM) as ammonium counter anion resulted in 94% loading compared with 93% when the counter anion of 500 mM ammonium is 250 mM of sulfate., following 4 days of storage at a temperature of 5° C.

### Example 2

### Preparation of Remotely Loaded Doxorubicin Liposomes

In this stage the efficiency of remote loading encapsulation and encapsulation stability were studied. Liposomes were prepared as described above. Briefly, the liposomes were made in four steps, 1) formation of liposomes containing ammonium counter ion, 2) liposome downsizing, 3) removal of medium ammonium salt for the creation of ammonium salt gradient, and 4) doxorubicin remote loading. All formulations were made from HSPC:Cholesterol:PEG-DSPE mole ratio of 54.5:41:4.5, briefly the lipids were hydrated and suspended in the various ammonium ions to form MLV. The MLV were downsized by extrusion followed by dialysis to remove external ammonium salt and form the gradient, finally drug was remote loaded into the gradient liposomes.

TABLE 4
Doxorubicin encapsulation efficiency using various ammonium counter ions

| | Counter | | | Encapsulation % |
|---|---|---|---|---|
| Ammonium | anion Conc. | Ammonium | Encapsulation | Short stability 5° C. |
| counter anion | (mM) | salt pH | % (T0) | After one week |
| Lactate | 250 | 4.5 | 18.37 | N/D |
| Lactate | 250 | 5.5 | 23.42 | N/D |
| Lactate | 500 | 6.2 | 101.95 | 48.68 |
| Pyruvate | 500 | 6 | 87.64 | 15.38 |
| Maleate | 500 | 5.5 | 22.55 | N/D |
| Maleate | 250 | 5.5 | 36.23 | N/D |
| Glycolate | 500 | 5.5 | 41.19 | N/D |
| Methanesulfonate | 500 | 5.53 | 106.32 | 110.66 |

As shown in Table 4, only liposomes made with ammonium methanesulfonate (“PLDMS Pyruvate and lactate anions demonstrated efficient encapsulation of doxorubicin at zero time, post production. However, preformed pegylated nano-liposomes having trans-membrane ammonium lactate and ammonium pyruvate gradients showed, encapsulation instability due to fast doxorubicin leakage and therefore, only preformed pegylated nano-liposomes having trans-membrane ammonium salts of sulfonic acid derivatives demonstrated high (>90% efficiency) and long term encapsulation stability.

### Example 3

### Stability of PLDMS Liposomes

Ammonium methanesulfonate liposomes (PLDMS) with different ammonium methanesulfonate gradient concentrations were stored in stability chamber at 5° C. (+/−) 1° C. in type III amber glass vials. Vials were tested at various time points and the PLD-MS product tested for drug content and encapsulation, and physical parameters as shown below in Tables 5 and 6.

TABLE 5
Chemical and encapsulation stability of doxorubicin (2 mg/mL) pegylated
liposomal doxorubicin remote loaded via trans-membrane of ammonium salts
of various selected sulfonic acids derivatives stored at 5° C.

| | | | Alkyl/aryl sulfonic | Doxorubicin | | |
|---|---|---|---|---|---|---|
| Time | | | acid derivative intra- | chemical | | External |
| point | | | liposome concentration | stability | encapsulation | medium |
| (month) | Formulation | Alkyl/aryl | (mM) | (%) | (%) | pH |
| 13.5 | DOXMS001- | Methane | 500 | 100.91 | 96.59 | 6.8 |
| | 100718 | sulfonate | | | | |
| 3 | DOXMS052- | Methane | 500 | 99.87 | 100.07 | 6.8 |
| | 110116 | sulfonate | | | | |
| 3 | DOXMS003- | Methane | 250 | 98.52 | 97.21 | 6.8 |
| | 101010 | sulfonate | | | | |
| 3 | DOXMS004- | Methane | 250 | 96.27 | 95.73 | 6.8 |
| | 101010 | sulfonate | | | | |
| 3 | DOXMS050- | Methane | 350 | 97.46 | 99.07 | 6.8 |
| | 110116 | sulfonate | | | | |
| 3 | DOXMS051- | Methane | 450 | 98.85 | 100.65 | 6.8 |
| | 110116 | sulfonate | | | | |
| 3 | DOXESA002 | Ethane | 250 | 83.19 | >95% | 6.8 |
| | | sulfonate | | | | |
| 3 | DOX3HPSA002 | 3-Hydroxy | 250 | 92.66 | >95% | 6.8 |
| | | Propane-1- | | | | |
| | | sulfonate | | | | |
| 3 | DOX4HBSA | 4- | 250 | 90.00 | >95% | 6.8 |
| | [symbol] | [symbol] | | | | |
| 2 | DOXESA003 | Ethane | 250 | 96.36 | >93% | 5.5 |
| | | sulfonate | | | | |
| 2 | DOX3HPSA003 | 3-Hydroxy | 250 | 95.72 | >94% | 5.5 |
| | | Propane- | | | | |
| | | 1-sulfonate | | | | |
| 2 | DOX4HBSA003 | 4-Hydroxy | 250 | ≈100% | >93% | 5.5 |
| | | Benzene | | | | |
| | | sulfonate | | | | |
| 2 | DOXMSA001 | Methane | 250 | ≈100% | >95% | 5.5 |
| | | sulfonate | | | | |
| 2 | DOXMSA00 | Methane | 500 | 97.34 | >95% | 5.5 |
| | [symbol] | [symbol] | | | | |
| 1 | Dox-G* | Glucuron | 250 | 77 | 100 | 5.5 |
| 3 | Dox026** | Sulfate | 250 | 96.85 | 100.76 | 5.5 |

*Dox-G refers to liposomes comprising doxorubicin made with ammonium glucuronate gradient
**Dox026 refers to liposomes comprising doxorubicin made with ammonium sulfate, similar to Doxil ® [symbol]
indicates data missing or illegible when filed

TABLE 6
PLDMS properties at various ammonium anion concentrations

| | | | | | | methane- |
|---|---|---|---|---|---|---|
| | | | Size & | Size & | Zeta | sulfonic |
| | pH | pH | PDI | PDI | Pot. | acid concen- |
| | (Zero | (3 | (Zero | (Three | (Zero | tration |
| Formulation | time) | months) | Time) | Months) | time) | (mM) |
| DOXMS001- | 6.67 | 6.87 | 82.78, | 84.51, | Not | 500 |
| 100718 | | | 0.045 | 0.030 | mea- | |
| | | | | | sured | |
| DOXMS052- | 6.61 | 6.56 | 80.27, | 81.58, | −9.46 | 500 |
| 110116 | | | 0.014 | 0.026 | | |
| DOXMS003- | 6.67 | 6.95 | 90.16, | 86.26, | Not | 250 |
| 101010 | | | 0.017 | 0.013 | mea- | |
| | | | | | sured | |
| DOXMS004- | 6.64 | 6.89 | 85.27, | 79.62, | Not | 250 |
| 101010 | | | 0.040 | 0.016 | mea- | |
| | | | | | sured | |
| DOXMS050- | 6.54 | 6.54 | 83.73, | 85.95, | −9.53 | 350 |
| 110116 | | | 0.031 | 0.038 | | |
| DOXMS051- | 6.58 | 6.58 | 90.84, | 91.47, | −9.88 | 450 |
| 110116 | | | 0.047 | 0.042 | | |
| DOX-MSA-02 | 6.8 | 6.7* | 91.6, | 92.4, | Not | 500 |
| | | | 0.04 | 0.05 | mea- | |
| | | | | | sured | |

*twelve months

### Example 4

### Aggregation/Precipitation/Crystallization of PLDMS Liposomes

CryoTEM was performed in order to study the state of aggregation of the doxorubicin in the liposomes. PLD and pegylated nano-liposomes having trans-membrane ammonium salts of the desired (alkyl and aryl) sulfonic acid derivatives after remote loading with doxorubicin (prepared as described above) were compared to Doxil® and to doxorubicin liposomes prepared with ammonium glucuronate gradient (see, e.g., WO 2005/046643). All liposomes were of the same size and identical lipid composition. Briefly, for cryo-TEM, a drop of the solution was placed on a carbon-coated holey polymer film supported on a 300 mesh Cu grid (Ted Pella Ltd), the excess liquid was blotted and the specimen was vitrified via a fast quench in liquid ethane to −170° C. The fast cooling preserves the structures present at the bulk solution and therefore provides direct information on the morphology and aggregation state of the objects in the bulk solution without drying. The samples were imaged at −180° C. using a FEI Tecnai 12 G2

Transmission Electron Microscope, at 120 kV with a Gatan cryo-holder maintained at −180° C.

As shown in FIGS. 2A-2G, ammonium glucuronate liposomes (DOXG) and PLDMS liposomes as described herein show identical cryo-TEM of spherical liposomes with no intraliposome drug crystallization or precipitation in contrast to Doxil® where crystallization inside liposomes many of which are non-spherical is apparent. As shown in Dox-ES (FIG. 2D) and Dox-3HPS (FIG. 2F) no intra-liposome drug crystallization or precipitation is observed while in Dox-4HBS (FIG. 2E), a similar image of intra liposome crystals similar to what is observed for Doxil®. Empty liposomes passively loaded with ammonium methanesulfonate 500 mm but without doxorubicin) presents in FIG. 2G show luck of intraliposome crystals.

The physical state of the doxorubicin-salt in the intraliposome aqueous phase was also confirmed by X-ray diffraction using wide angle and small angle X ray diffraction (WAXS and respectively (see Tables 7A, 7B and below).

### Example 5

### X-Ray Scattering Analysis

WAXS analysis was performed using a procedure developed by Dr. Raviv (The institute of Chemistry, Hebrew University of Jerusalem, Israel (HUJI). Briefly, the X-ray generator, MicroMax-007HF (Rigaku Corporation), is a rotating anode operating at 40 kV and 30 mA and has a copper target producing K_{α} photons with an energy of 8 keV (wavelength of 1.54 Å). The rotating anode is water-chilled by a refrigerated air-cooled system (Haskris, R075). A focused monochromatic beam is obtained using Confocal Max-Flux optics consisting of a CMF-12-100Cu8 focusing unit (Osmic Inc., a Rigaku Company). The beam continues into a vacuum flight path (ca. 15 Torr), which contains two slits; fully motorized, scatterless hybrid metal_Ge single-crystal slits (Forvis Technologies, Inc). The flight path is closed by a Kapton window; which causes a parasite peak at 4.1 nm⁻¹. The sample holder is placed immediately after the slits, and a MAR345 image-plate detector (Marresearch GmbH) is stationed at 250 mm of the sample. During Small Angle X-rays Scattering experiments (SAXS), after the sample, the scattered beam enters a large He-filled flight path (ca. 36 cm in diameter) before to be collected on the Mar345 image plate detector, placed about 1850 mm after the sample holder.

The details on the SAXS measurements are very similar to those of the WAXS (described above). The main difference is that the sample to detector distance is for the SAXS 1850 mm (instead of 250 mm for the WAXS measurements). The scattered beam is going through a He flight path to avoid air scattering over such a long distance.

The measurements were performed in a temperature-controlled sample chamber (Forvis Technologies Inc., CA) with 0.1° C. accuracy. After the temperature was changed to the desired value, a 1 hour time before the measurement was used to achieve thermal equilibration of the liposomal dispersion at the desired temperature. See, also, Nadler M. et al., Soft Matter (2011), 7, 1512-1523.

Sample Preparation.
1 Ammonium alkyl or aryl sulfonate was prepared by dissolving relevant sulfonic acid in water following by a titration with ammonium hydroxide to final pH=5.5.
2. Lipid mix was dispersed in ammonium alkyl or aryl sulfonate to form MLV (Multi Lamellar large Vesicles) followed by extrusion process for SUV (Small unilamellar Vesicles) to achieve ˜85-90 nm liposomes of homogenous uni-modal size distribution.
3. SUV's were subjected to dialysis for external Ammonium alkyl or aryl sulfonate followed by Doxorubicin encapsulation into SUV liposomes. Histidine buffer was added.

The characterization features of all different liposomal formulations used in this study are summarized in Tables 7A and 7B.

TABLE 7A
X-RAY and CRYO-TEM- formulations compositions

| | | | | Lipids | Doxorubicin |
|---|---|---|---|---|---|
| sample | formulation | ammonium counter | ammonium | concentration | concentration |
| # | name | ion and valency | conc. | (estimated) | (estimated) |
| 1 | DOXMSA003 | Methanesulfonate (−1) | 350 mM | ≈16 mg/ml | ≈2 mg/ml |
| 2 | DOX3HPSAA004 | 3-HydroxyPropane-1- | 350 mM | ≈16 mg/ml | ≈2 mg/ml |
| | | Sulfonate (−1) | | | |
| 3 | DOX4HBSAA004 | 4-HydroxyBenzene | 350 mM | ≈16 mg/ml | ≈2 mg/ml |
| | | Sulfonate (−1) | | | |
| 4 | DOXESA003 | EthaneSulfonate (−1) | 350 mM | ≈16 mg/ml | ≈2 mg/ml |
| 5 | LIPODOX | Sulfate (−2) | 500 mM | ≈16 mg/ml | ≈2 mg/ml |

TABLE 7B
X-RAY diffraction and CRYO-TEM- physical properties (for
the formulation characterization see Table 7A above)

| Sam- | lipo- | Cryo-TEM | Cryo-TEM | | | |
|---|---|---|---|---|---|---|
| ple | some | (doxorubicin | (liposome | X-Ray | X-Ray | X-Ray |
| # | size | shape) | shape) | (4° C.) | (37° C.) | (60° C.) |
| 1 | ≈90 nm | No | Spheric | No | No | No |
| | | crystals | | crystals | precipi- | precipi- |
| | | | | | tation | tation |
| 2 | ≈90 nm | No | Spheric/ | No | No | No |
| | | crystals | ellipsoid | crystals | crystals | precipi- |
| | | | | | | tation |
| 3 | ≈90 nm | crystals | Ellipsoid | precipi- | No | No |
| | | | | tation | precipi- | precipi- |
| | | | | | tation | tation |
| 4 | ≈90 nm | No | Spheric | No | No | No |
| | | crystals | | precipi- | precipi- | precipi- |
| | | | | tation | tation | tation |
| 5 | ≈90 nm | crystals | Ellipsoid | precipi- | precipi- | No |
| | | | | tation | tation | precipi- |
| | | | | | | tation |

Furthermore, as shown in FIG. 5A, for the samples 1 (Dox-MS), 2 (Dox-3HPS) and 4 (Dox-ES), liposomes remote loaded with doxorubicin by trans-membrane gradients of ammonium salts of (Dox-MS), 2 (Dox-3HPS) and 4 (Dox-ES), no diffraction peak related to the crystallization of doxorubicin was observed. The WAXS scattering curves of those samples were, in this respect, very similar to the scattering curves of the same liposomes before doxorubicin remote loading (drug free liposomes). However, in samples 3 (Dox-4HBS) and 5 (Dox-sulfate=Doxil®), a doxorubicin crystallization peak at ca. 2.3 nm⁻¹ is observed, similarly to what was reported long ago by Lasic et al FEBS Letters 1992, 312, 255-25.

In addition, the lipid tails in the liquid (liquid disordered or liquid ordered) phase, contribute to the signal with a weak peak around 15 nm⁻¹. See, e.g., Spaar et al. Biophys. J. 85(3) 1576-1584. Comparing the curves of loaded and empty liposomes, the presence of the drug apparently decreases the level of order of the lipid tails, suggesting that doxorubicin interacts with the membrane lipids. However, this effect is present in all formulations and is not related to the intraliposome drug crystallization.

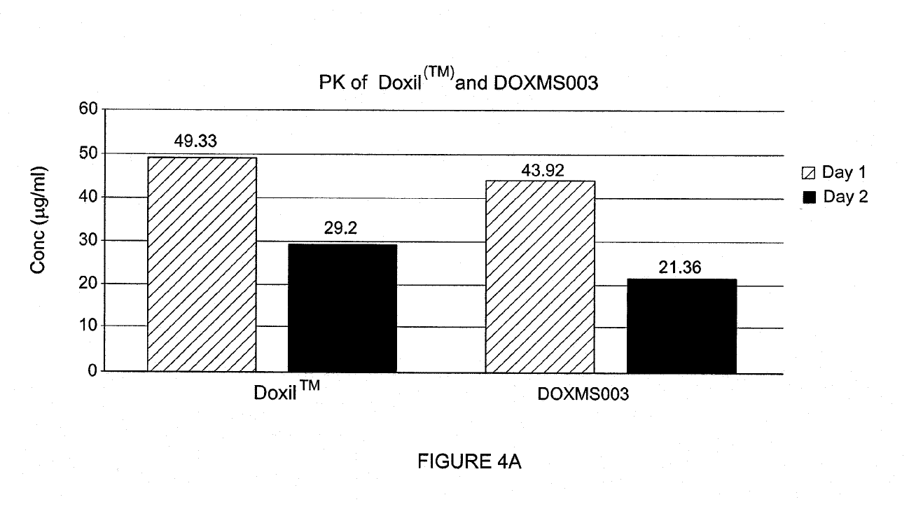

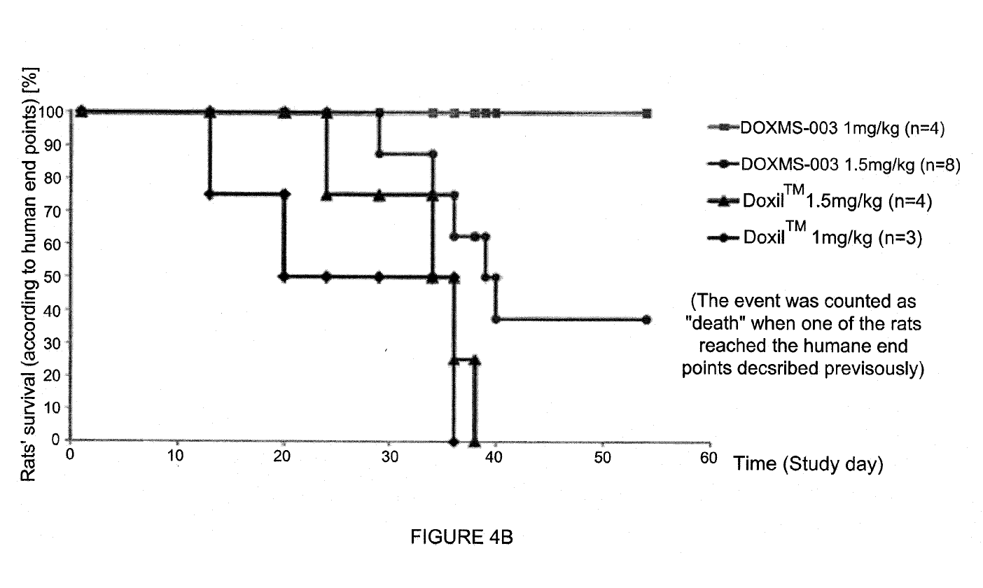

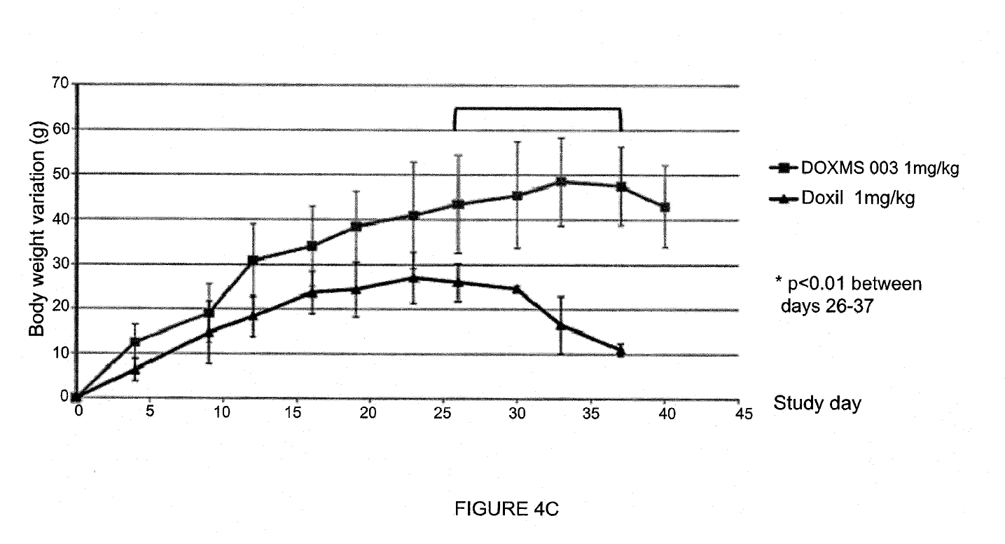

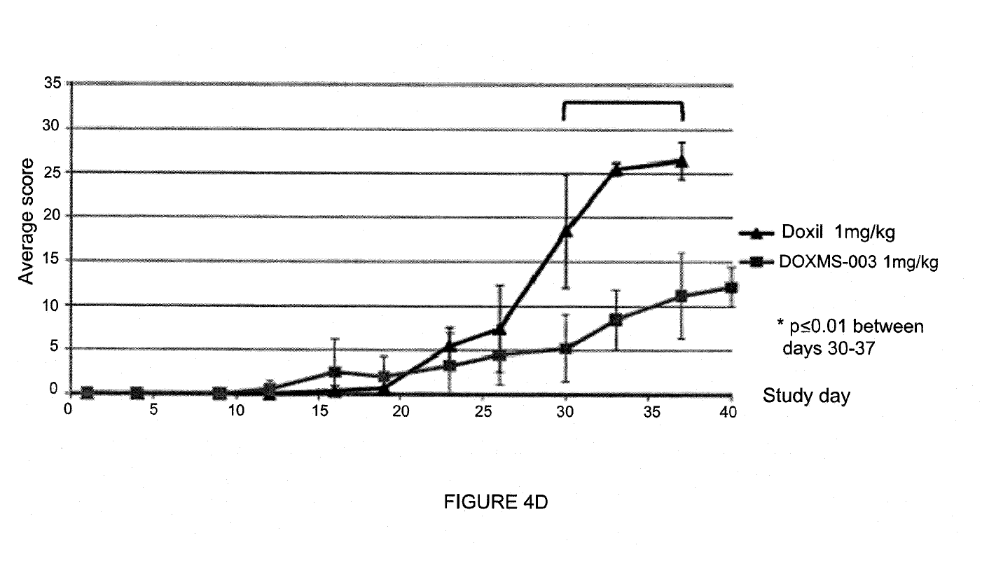

As shown in FIG. 5D, for samples 3 (Dox-4HBS) and 5 (Dox-sulfate), at low wave vector—q-, the scattering intensity decreases with q, while it increases in all the other scattering curves-samples 1, 2 and 4. This behavior is correlated to the presence of the doxorubicin crystalline peak, marked by an arrow. The marked peak of sample 3, is weaker but at the same position than the peak of sample 5. This indicates that the doxorubicin crystallization affects the liposome shape (Brzustowicz et al. (2005) J. Appl. Cryst. 38:126-131) and is another evidence that it forms crystals inside the liposomes. These results are in agreement with the cryo electron micrographs of the different formulations (FIG. 4E—Dox-4HBS, FIG. 2A—Dox-sulfate)

FIG. 5B presents the effect of temperature on the WAXS spectra of samples 1 (Dox-MS), sample 3 (Dox-4HBS) and sample 5 (Dox-sulfate). Samples 2 (Dox-3HPS) and 4 (Dox-ES) present exactly the same features than sample 1 (Dox-MS). For sample 5, the crystalline doxorubicin phase is present at both 4° C. and at 37° C., but not at 60° C. and in sample 3, the peak is present only at 4° C. Even at 4° C., samples 1, 2 and 4 don't present the doxorubicin crystalline peak. Those results mean that the crystallization temperature of the intra-liposome doxorubicin depends on the ammonium-counter anion salt used for the remote loading. This counter ion also makes the intra-liposome doxorubicin salt.

FIG. 5C, show a small angle X ray scattering (SAXS) in which the doxorubicin crystal peak is observed in both Mimicry of Doxil® and commercial Doxil®, as already presented in FIG. 2A, while for liposomes loaded with DOXMS, doxorubicin does not show any crystallization signs.

### Example 6

### In Vitro Release Profile

Doxil® and pegylated nano-liposomes having trans-membrane ammonium salts of the desired alky and aryl sulfonic acid derivatives after remote loading with doxorubicin (prepared as described above) were diluted to a final concentration 40 μg/ml of lipids in 50 mM ammonium sulfate buffer. The cation exchanger Dowex™-50 was added to each sample in order to act as a sink that bind irreversibly all the released doxorubicin (Druckmann et al. (1989) Biochem. Biophys. Acta 980:381-384) which thereby preventing its re-uptake by the liposomes (such re-uptake occurs as liposomes retain part of their trans-membrane ammonium gradient). Samples were incubated at 37° C. with shaking. At each time point, the samples were removed from incubation, vortexed, and then centrifuged at 4000 rpm for 5 minutes. Aliquots from the resulting supernatant were taken and analyzed on a BioTek spectrophotometer (Biotek-Synergy 4, Vermont, USA) using absorbance at a wavelength of 480 nm. The samples were then vortexed and returned to incubation. The various liposomal doxorubicin samples were incubated over 48 hours and release of doxorubicin was tested periodically.

Release Procedure:

5 ml solution containing Pegylated liposomal doxorubicin 2 mg/ml: 50 mM ammonium sulfate with 20 mM Histidine solution (pH=7.3±0.2), 1:50 respectively, was placed on 150 mg Dowex (30 mg/ml) following incubation for 24 hours at a temperature of 37° C. Mixing rate was 50 RPM. Aliquots were removed in zero time (T-0) and following 3, 5 and 24 hours, centrifuged and analyzed using Biotek for absorbance at a wavelength of 490 nm. Achieved absorbance was compared to T-0 absorbance, for doxorubicin release rate from the liposome.

Pegylated liposomal doxorubicin (2 mg/ml doxorubicin), remote loaded into pegylated nano-liposomes containing one of the 350 mM sulfonate derivatives ammonium salt (prepared by titration of the sulfonic acid derivative to pH=5.5±0.1 by ammonium hydroxide to form ammonium-sulfonate derivative salt)—external storage medium buffer pH is 6.02 compared with DOXNP-250 mM sulfate (prepared by remote loading of the liposomes containing 250 mM ammonium sulfate), external storage buffer pH was 6.50. These formulations were evaluated and compared for release tests.

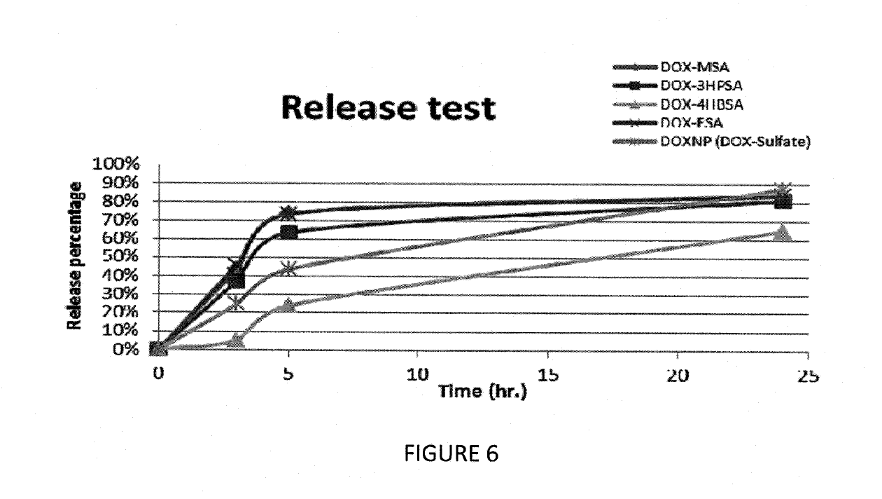

As shown in FIG. 6, doxorubicin release rate when doxorubicin was remotely loaded into pegylated liposomes to form Dox-MS was equal to Dox-ES and very similar to Dox-3HPS, following three and five hours of incubation as describe above. All these showed faster release rate than liposomes having trans-membrane ammonium sulfate remote loaded Doxil®-like liposomes. However, doxorubicin release rate when doxorubicin was remotely loaded into pegylated liposomes to form Dox-4HBS was unexpectedly even slower than of Doxil®-like liposomes.

### Example 7

### In Vivo Pharmacokinetics (PK) and Biodistribution (BD)

A total of 31 Balb/c mice were injected intravenously (I.V.) with a single dose of Doxil® or the of various PLDMS shown in Table 8 below. At defined time-points (see Table 8 below, for composition of each formulation see Tables 5 and 6), mice of each group were euthanized with CO₂ and terminal blood was withdrawn from the retro-orbital sinus and collected in labeled K3EDTA tubes.

TABLE 8
Injection and euthanization schedule

| Injected Formulation | Total # of mice | Time points of euthanization (# of | |
|---|---|---|---|
| DOX-046 | 10 | 24 hours | (5 mice) |
| | | 48 hours | (5 mice) |
| DOX-050 | 10 | 24 hours | (5 mice) |
| | | 48 hours | (5 mice) |
| Doxil ® | 10 | 24 hours | (5 mice) |
| | | 48 hours | (5 mice) |
| free doxorubicin | 7 | <1 min | (4 mice) |
| | | 10 min | (3 mice) |

The blood samples were then immediately subjected to plasma separation procedure (4000 rpm for 10 minutes at 4° C.). The plasma, red blood cells fraction and whole blood of each mouse has been collected in labeled CryoTubes and stored at −80° C. pending analysis.

Doxorubicin was extracted from the samples as follows. The samples were diluted in acid isopropyl alcohol (A-IPA) and vortexed 30 seconds then centrifuged 14K RPM for 5 minutes for plasma protein precipitation. From the upper phase, 100 μl of the plasma diluted in A-IPA were diluted in 900 μl mobile phase for analysis and the contents of extracted doxorubicin was determined using fluorescence HPLC (as described in Gabizon et al. (1993) Pharm. Research 10(5):703-708).

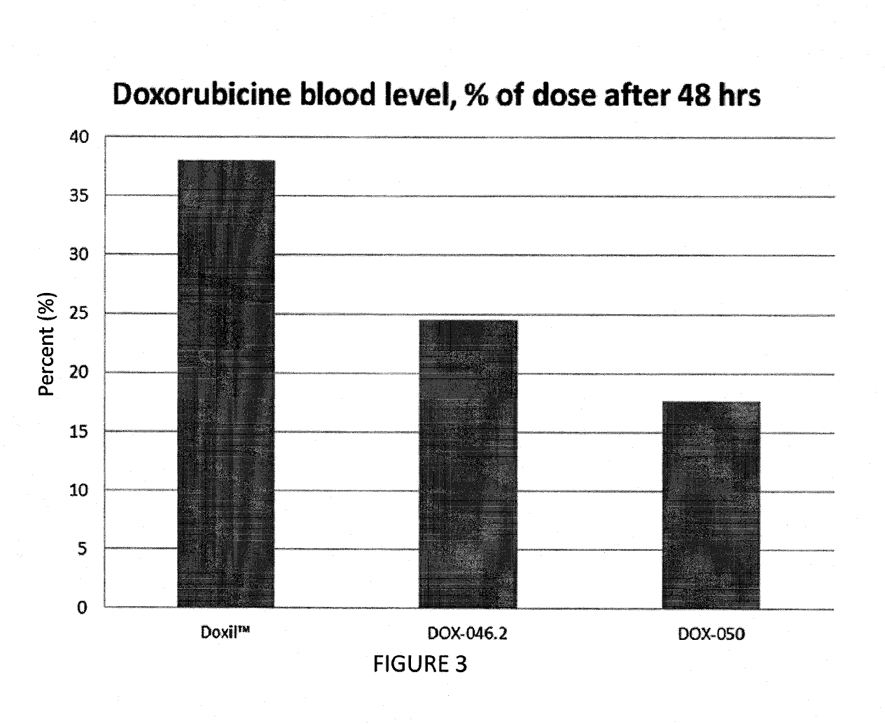

As shown in FIG. 3, PLDMS-treated animals had much lower drug levels in blood after 48 hours in comparison to Doxil® and blood levels of mice injected with free doxorubicin was below limit of detection practically zero.

The above experiments were repeated with PLDMS liposomes (“DOX-046.3) made using a 250 mM ammonium methanesulfonate gradient. As shown in FIG. 4A, PLDMS liposomes show lower drug levels in blood after 24 and 48 hours in comparison to Doxil® demonstrating shorter residence time of doxorubicin in blood and hence likely leading to fewer (or reduced) side effects.

The pharmacokinetics (PK) and a biodistribution (BD) of the liposomes were also studied in order to compare PLDMS with commercial Caelyx was performed. The details of the procedure are described below and in Table 9.

TABLE 9
Test items details

| | Sample Details | |
|---|---|---|
| | LC-100/DOXMS/PLDMS | Caelyx ® |
| Complete product | DOXMS | Doxorubicin HCl liposome |
| name | Doxorubicin 2 mg/ml | injection (Caelyx ®) |
| | | Doxorubicin 2 mg/ml |
| | | By Ortho Biotech Products |
| | | L.P., USA |
| Batch # | DOXMS-010-120708 | 101371803 |
| Manufacturing | Aug. 7, 2012 | December 2010 |
| date | | |
| Expiry date | — | August 2012 |
| Appearance | Orange to red translucent | Orange to red translucent |
| | solution | solution |
| Storage conditions | 2-8° C., protected from | 2-8° C., protected from |
| | light | light |
| Ammonium salt | Ammonium Methane | Ammonium sulfate |
| concentration | sulfonate 350 mM | 250 mM |

### Study Procedure

### General

A total of one hundred and ten (110) female Balb/c mice were injected intravenously (I.V.) with a single dose of LC100 or Caelyx® that was equivalent to 200 μg DOX (55 mice per tested group). At specified time-points, five mice of each group were sacrificed and blood was immediately collected and subjected to plasma separation procedure. Immediately following blood collection the mouse was perfused with approximately 10-15 mL of saline then the following organs were collected separately in labeled CryoTubes and immediately subjected to cryopreservation in liquid nitrogen: liver, heart, spleen, kidneys, lungs, brain and ovaries. The organs were transferred into labeled boxes at −80° C. pending analysis.

By a controlled procedure, doxorubicin was extracted from the plasma samples and the content of extracted DOX was determined using fluorescence-HPLC procedure as described below.

For the biodistribution study, doxorubicin was extracted from liver and heart according to the procedure described in paragraph 00114 and 0016 below and the content of extracted DOX was quantified fluorometrically (λ_{excitation} 485 nm and λₑₘᵢₛₛᵢₒₙ 620 nm). In order to compare the values to the organ biodistribution of free doxorubicin, Balb/c female mice of similar age and body weight to the mice used in this study were injected using exactly the same procedure with 200 μg of doxorubicin hydrochloride in a separate experiment. The blood and organ collection were performed following the procedures described below. The collected organs of mice treated with doxorubicin hydrochloride were treated and analyzed together with the organs of mice treated with Caelyx® and LC100.

### Preparation of Blood Samples

At each time point five mice of each group were sacrificed with CO₂ and terminal blood was immediately withdrawn from the retro-orbital sinus and collected in labeled 0.5 mL K₃EDTA blood collection tubes (Mini Collect, Greiner-bio-one, Austria). The blood was centrifuged at 4000 rpm (2060 g) for 10 minutes. The plasma was collected in labeled CryoTubes and cryopreserved immediately in liquid nitrogen and then transferred into labeled boxes and stored at −80° C. pending analysis.

### Preparation of Organs

Immediately following blood collection, the mice were perfused with 10 to 15 mL saline (according to need, until the liquid coming out of the incision in the heart was clear) and the following organs were collected separately in labeled CryoTubes and immediately subjected to cryopreservation in liquid nitrogen: liver, heart, spleen, kidneys, lungs, brain and ovaries. The organs were then transferred into labeled boxes at −80° C. pending analysis.

### Extraction and Analysis Procedures

### Plasma Samples

Plasma samples were delivered by the in vivo pharmacologist to the QC department accompanied with a controlled delivery form (“Collection of blood samples for analysis” and “Collection of organs samples for analysis” forms). Extraction procedure and measurement of DOX content from plasma samples were carried out by QC personnel according to protocol “Analysis protocol for PK study PK003-LC100-120904.”

### Organ Samples

Total liver and heart doxorubicin was quantified using a method similar to that of Charrois & Allen (2004) Biochim Biophys Acta. 1663(1-2):167-77. Organs samples (liver from time-points T=8 h, 24 h and 48 h after injection as well as heart samples of time-point T=8 h after injection) were left at room temperature for thawing then weighed and cut to small pieces with a scalpel blade. The tissues' pieces were firstly homogenized in 2 ml pure water per gram tissue. This was followed by addition of acidified isopropanol (0.075N HCL in 90% isopropanol 10% water) to a final volume of 10 ml per gram tissue and homogenization using a Polytron PT2100, Kinematica AG, Switzerland) homogenizer. For heart samples, DOX extraction was done in the presence of 120 μl of hydrogenated Triton X-100. The tubes were vortexed thoroughly, and the homogenates were left overnight at 4° C. The next day, the tubes were warmed to room temperature, vortexed for 5 min, centrifuged at 15,000 g for 20 min, and doxorubicin was quantified spectrofluorometrically from the supernatant using the BioTek, Synergy4 using excitation filter of λ_{excitation} 485+/−20 nm and λₑₘᵢₛₛᵢₒₙ 620+/−40 nm. To correct for nonspecific background fluorescence, the samples were analyzed using a standard curve containing tissue extracts derived from drug-free mice. The values were then corrected for any potential quenching of the fluorescence by using a standard curve in the tissue extraction buffer.

To correct for extraction efficiency a calibration curve in buffer and in tissues spiked with doxorubicin and extracted as above were compared. In all cases doxorubicin extraction efficiency was >60% . All data were corrected to extraction efficiency at the right drug concentration.

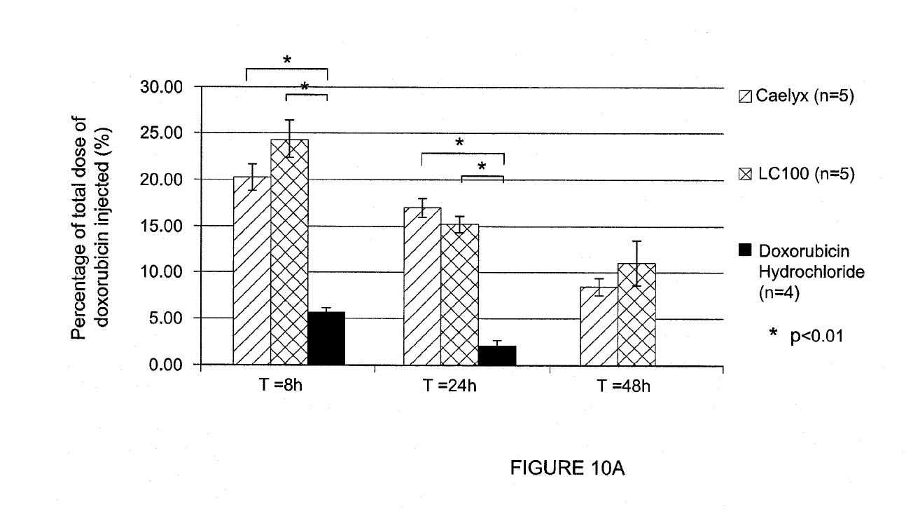

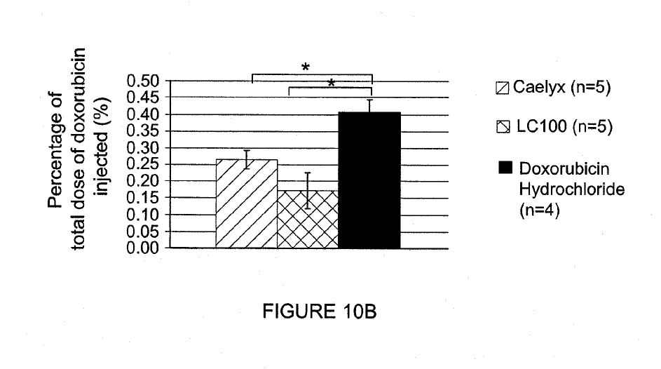

The results are summarized in FIGS. 10A and 10B (and Tables 7 and 8).

### Summary and Conclusions of PK and BD Studies

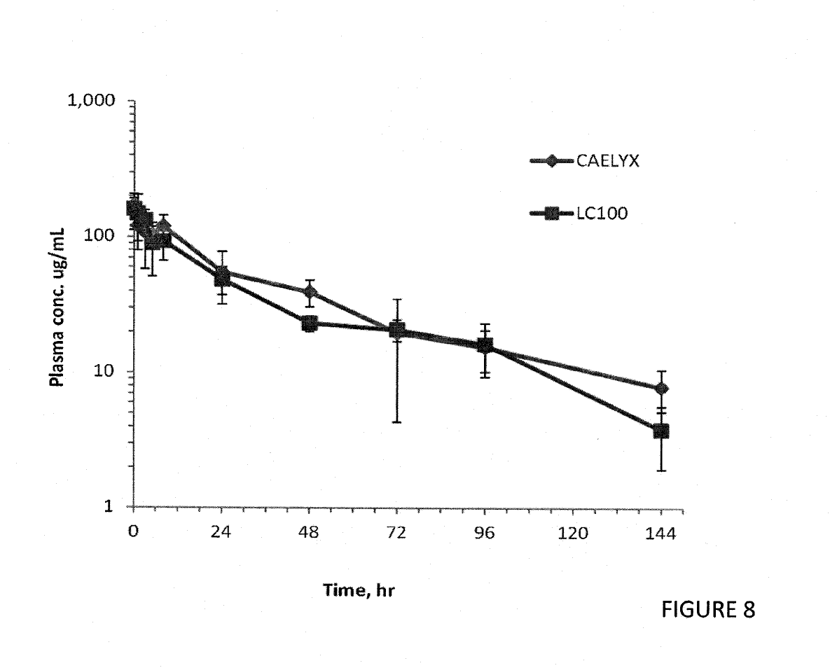

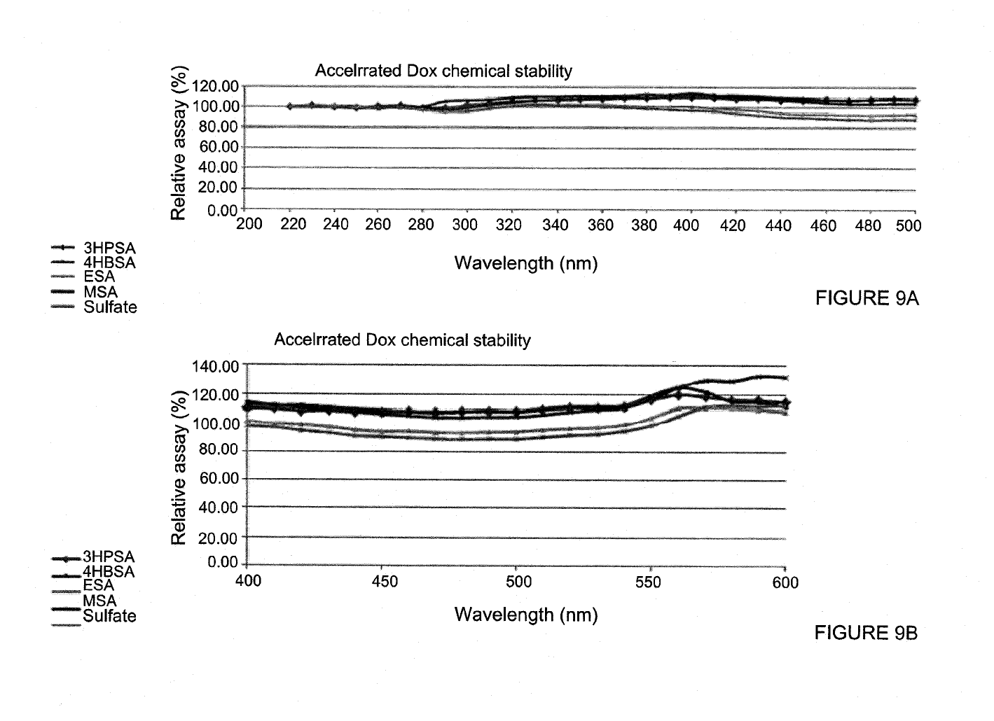

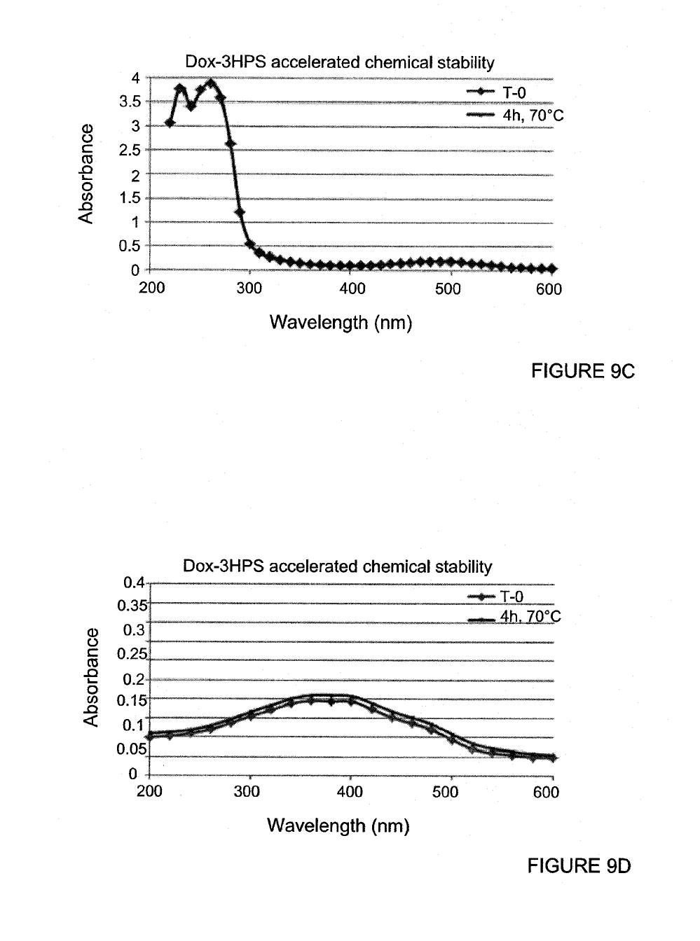

Intravenous administration of equal doses of LC 100 and Caelyx to healthy normal mice resulted in similar prolongation of the circulation time with somewhat lower doxorubicin plasma levels at 48 (2 days) and 144 (1 week) levels (FIG. 8). Table 10 demonstrates that LC100 is having lower AUC (˜30%), Lower MRT and t_{1/2} (85%) than Caelyx without affecting much Vss. This may explain the similar therapeutic efficacy of the two liposomal doxorubicin preparations described in Example 9 below as well as the better tolerability described in example 8 below. The data of represent the mean±S.E. of triplicate aliquots from four to five mice and are expressed as a percentage of injected dose.

No statistical difference was found between the dose of doxorubicin derived from Caelyx® or LC100 accumulating in the liver at 8, 24 or 48 hours after injection of the compounds (FIG. 10A). However, liver of mice injected with one of the two liposomal formulations described above show a statistically higher doxorubicin level than livers of mice injected with equal dose of free doxorubicin at all time-points studied.

In the heart 8 hours after injection, there was a statistically significant higher accumulation of doxorubicin in mice injected with free (non-liposomal) doxorubicin (doxorubicin hydrochloride) compared to mice injected with LC100 and Caelyx® (FIG. 10B). Although in the heart it seems that the level of doxorubicin derived from LC100 tends to be lower than the level reached after equal dose of Caelyx®, this difference was not statistically significant.

Together, the data shows that LC100 seems to have a similar profile of biodistribution to Caelyx® and should therefore protect the heart from cardio toxicity in the same way (or even better) than Caelyx® (Doxil®) from doxorubicin related cardio-toxicity (Barenholz JCR 2012 160, 117-134).

TABLE 10
The outcomes of non-compartmental pharmacokinetic analysis of the
average doxorubicin plasma concentration vs. time data

| | | | | % of |
|---|---|---|---|---|
| Parameter | Unit | CAELYX | LC100 | CAELYX |
| Lambda _z | 1/h | 0.0165 | 0.0196 | 119 |
| t_{1/2} | h | 41.9 | 35.4 | 84 |
| Tₘₐₓ | h | 0.017 | 0.017 | 100 |
| Cₘₐₓ | μg/ml | 171 | 160 | 94 |
| C₀ | μg/ml | 172 | 160 | 93 |
| Clast_obs/Cmax | — | 0.0457 | 0.0237 | 52 |
| AUC 0-t | μg*h/ml | 5151 | 4356 | 85 |
| AUC 0-inf_obs | μg*h/ml | 5622 | 4549 | 81 |
| AUC 0-t/0-inf_obs | — | 0.916 | 0.958 | 105 |
| AUMC 0-inf_obs | μg*h²/ml | 293073 | 201504 | 69 |
| MRT 0-inf_obs | h | 52.1 | 44.3 | 85 |
| V₀ | mL | 1.16 | 1.25 | 107 |
| Vss_obs | mL | 1.85 | 1.95 | 105 |
| Vz_obs | mL | 2.15 | 2.24 | 104 |
| CL_obs | mL/h | 0.0356 | 0.0440 | 124 |

### Example 8

### Comparison of Doxil® (Caelyx®) and LC100 on PPE Side-Effect and Well-being in Rats

Briefly, twenty Sprague-Dawley male rats were injected intravenously (IV) with PLDMS (“DOXMS003”) or Doxil®, either twice weekly (every 3 or 4 days) or every 5 days, for 40 days. The rats injected twice a week received a dose of 1 mg/kg at every injection (total of 12 mg/kg) and the rats injected every 5 days were injected at 1.5 mg/kg (total dose of 13.5 mg/kg). The first day of dosing was designated Study Day (SD) 1.

The rats were checked before each injection for clinical symptoms of PPE. The clinical symptoms observed on rats were scored according to a six-point severity grading system on six different area of the body, the maximum lesion score at any scoring time point is thus 36. All the rats marked as “dead” on the FIG. 4B were sacrificed because they reached the criteria of “humane” endpoints, no rat died spontaneously.

As shown in FIG. 4B, all the rats injected with Doxil® (at 1 or 1.5 mg/kg) were euthanized before the end of the experiment, most of them between the study days 34 and 38. In contrast, all the rats injected with PLDMS (“DOXMS003”) at 1 mg/kg survived throughout the study and until the end of the recovery period, and fifty percent of the rats injected with DOXMS003 at 1.5 mg/kg survived until the end of the injection period (with one euthanized before the beginning of the recovery period).

In addition, as shown in FIG. 4B, animals that received PLDMS (“DOXMS003”) at 1 mg/kg had a significantly higher body weight than the rats injected with Doxil® at the same dosage. The rats injected with the drugs at 1.5 mg/kg showed no statistical difference of body weight.

In terms of PPE, as shown in FIG. 8, rats injected with PLDMS (“DOXMS003”) at 1 mg/kg had a significantly lower score of PPE symptoms than the rats who were administered with Doxil® at 1 mg/kg, from study day 30 and until the end of the injections. Rats injected with Doxil® or PLDMS (“DOXMS003”) at the higher dose of 1.5 mg/kg had high, but a similar scoring of the PPE symptoms.

The rats that received PLDMS (“DOXMS003”) also appeared to be in better shape and suffering less than their peers who were treated with Doxil®. Specifically, mice treated with LC100 did not show asthenia (lack or loss of strength and energy; weakness), that mice treated with Doxil® showed. Notably, asthenia is the most common all-grade adverse reaction (40%) reported by patients with recurrent ovarian cancer treated with Doxil®.

In sum, PLDMS (“DOXMS003”) administered at 1 mg/kg show much lower PPE score and much better quality of life in term of general physiology (body weight, appearance) and clinical signs when compared with the rats that were injected with commercial Doxil® at the same regimen.

### Example 9

### In Vivo Anti-Tumor Effects in A549 Tumor Model in Nude Mice

Approximately 5 million AA549 lung cancer tumor cells were inoculated subcutaneously (s.c.) in the back of 5 week old NUDE-Hsd: Athymic mice (Harlan Laboratories, Jerusalem, Israel). Tumor weights were determined according to the equation ½×length×width 2 using direct caliper measurements (Euhus et al. (1986) Surg. Oncol. 31(4) 229-234; Tomayko et al. (1989) Cancer Chemother. Pharmacol. 24(3):148-154).

When the tumor reached ˜750 mg in weight, animals were administered a single injection (intravenous) either Doxil® or PLDMS drugs at a dose of 8 mg doxorubicin per kg. Doxorubicin concentration in Doxil® and PLDMS was identical ˜2 mg/ml. Forty eight hours post injection, animals were sacrificed and tumors were excised and sectioned.

Histopathological studies included staining for mitochondrial enzyme activity by incubating representative tissue sections for 30-45 min in 2% 2,3,5-triphenyl tetrazolium chloride (TTC) at room temperature to identify irreversible nonspecific cellular injury as described in Liszczak et al. (1984) Acta Neuropathol. 65(2):150-157). Gross measurements of tumor destruction were performed on both TCC-stained and unstained sections, and photographed. The extent of visible coagulation was measured with the image processing and analysis software ImageJ (NIH, Bethesda, Md.). Coagulation area was measured by precise selection of the white zone in the stained tumor section under high zoom.

The analysis showed largely similar levels of tumor coagulation of 367+/−65 arbitrary units for Doxil® and 423+/− units for PLDMS, where the higher the score, the greater the therapeutic effect.

### Example 10

### Therapeutic Efficacy Studies: OVCAR-3 Ovarian Adenocarcinoma Xenograft Model of Athymic Nude Mice

To evaluate the anti-tumor therapeutic efficacy and tolerability of the liposomal doxorubicin formulations based on ammonium methanesulfonate remote loading (DOX-MS=PLDMS) LC3-PLDMS-2 and LC4-PLDMS-5 in comparison to free (non-liposomal) doxorubicin in NIH: OVCAR-3 ovarian adenocarcinoma xenograft model of athymic nude mice.

Female athymic nude mice were inoculated subcutaneously in the left flank with a suspension of 5×10⁶ human OVCAR-3 ovarian adenocarcinoma cells (200 □L injection volume) and monitored for tumor growth. Animals were selected, randomized into four treatment groups (n=8 per group) with a balanced tumor size of ˜100 mm³ according to the study design (Table 9) below.

TABLE 9
Study Design

| Group | | Dose level | |
|---|---|---|---|
| number | Description | (mg/kg) | No. of animals |
| G1 | Control | 0 | 8 |
| G2 | Doxorubicin | 8 | 8 |
| | HCl | | |
| G3 | LC3- | 8 | 8 |
| | PLDMS-2 | | |
| G4 | LC3- | 8 | 8 |
| | PLDMS-5 | | |

G1 animals served as vehicle control and received 5 ml/kg saline. 2, G5 and G6 animals were treated with 8 mg/kg doxorubicin HCl, LC3-PLDMS-2 (PLMDS-2 are liposomes containing 250 mM ammonium methanesulfonate (MS) and remote loaded with doxorubicin (group 5 in the experiment), LC4 PLDMS-5 are liposomes containing 500 mM ammonium methanesulfonate (MS) and remote loaded with doxorubicin (group 6 in the experiment) (respectively, given intravenously (5 mL/kg dose volume) once weekly for 2 consecutive weeks. Body weight, general clinical observations and tumor size were monitored throughout the experimental period. Experimental groups were terminated when median tumor volume reached 2500 mm³. Endpoint parameters such as body weight change, % ILS, Median tumor volume, TGI, % T/C, RTV, LCK, Tumor growth delay, TVDT and TVDTD were calculated. Tumor volumes were measured twice weekly using a digital vernier caliper recording length (L=longest axis) and width (W=shortest axis), and volume was calculated in mm³ as L×W²/2. Groups reaching a median tumor volume of 2500 mm³ were terminated and the date of termination was used as the date of death for the purpose of survival calculations. The following parameters were calculated: median survival time (MST), percentage increased life span (% ILS) and median tumor volume (TV). Other measured, including median TV change; median tumor growth inhibition (TGI); % T/C; relative tumor volume (RTV); log cell kill (LCK); tumor growth delay; tumor volume doubling time (TVDT); and tumor volume doubling time delay (TVDTC), were derived from the median tumor volume measurements. Tumor growth regression was also plotted.

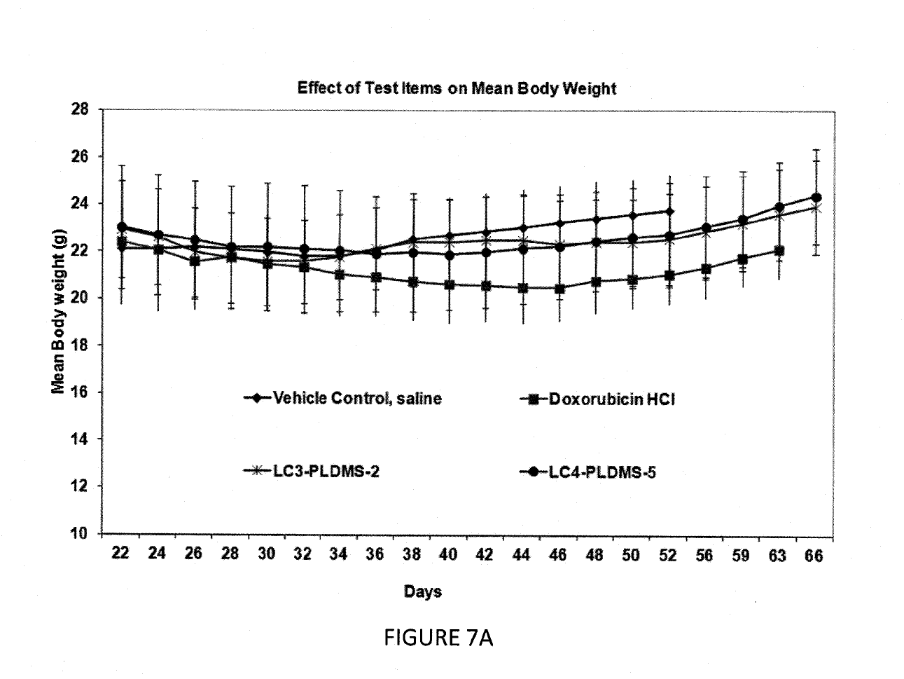

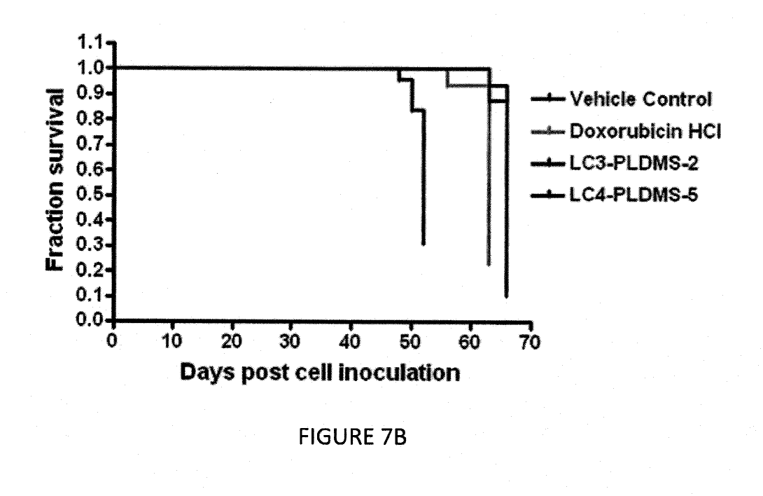

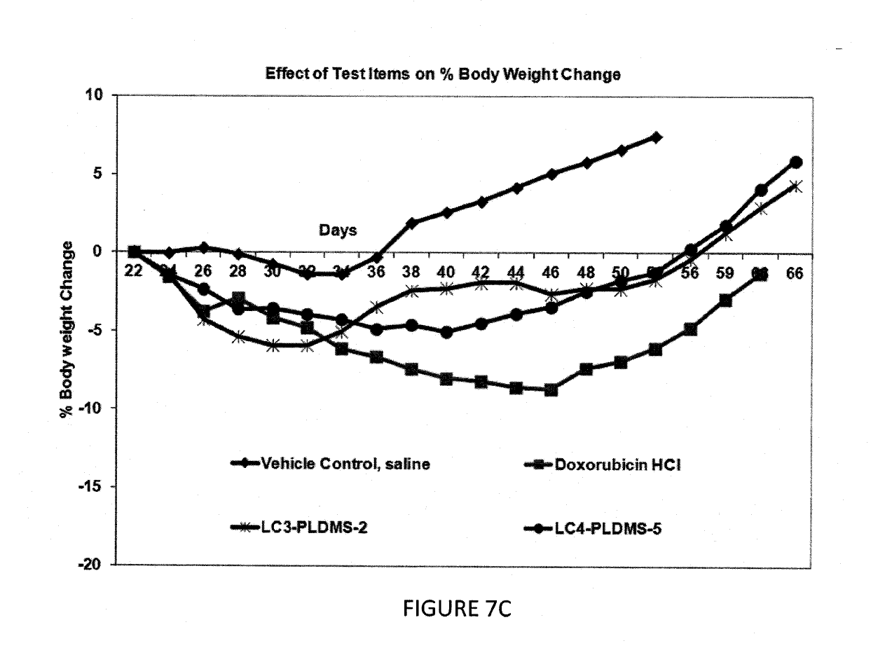

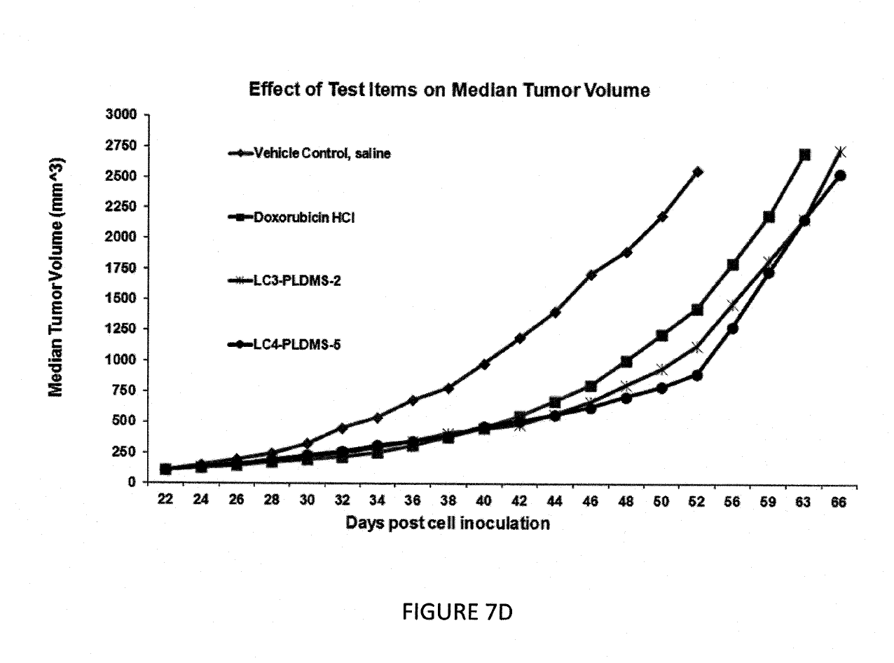

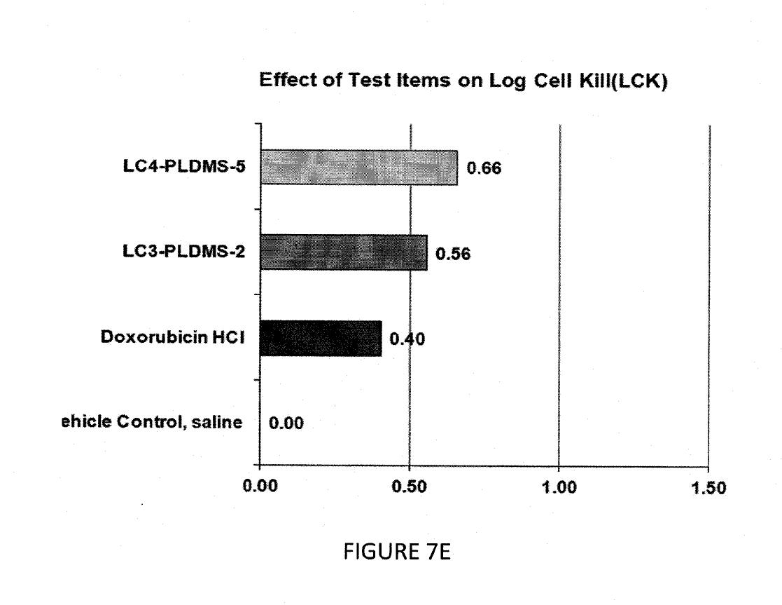

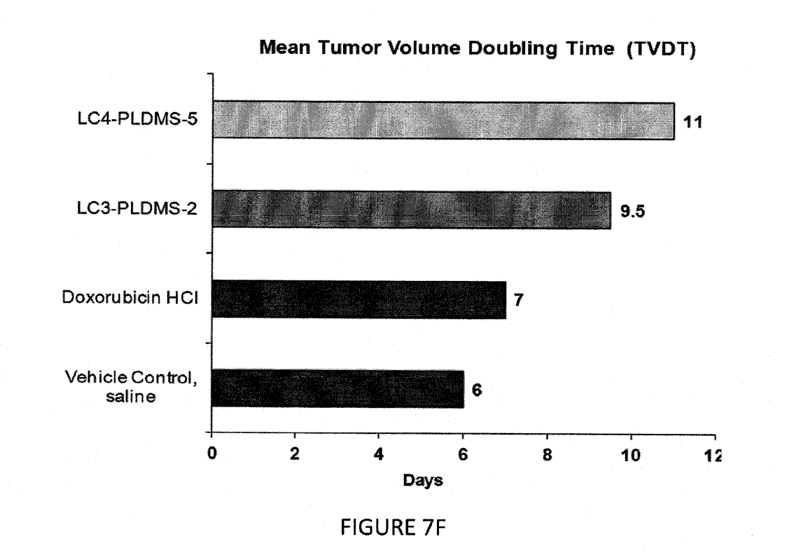

No clinical signs of toxicity were seen in either the LC3-PLDMS-2 or LC4-PLDMS-5 treatment groups and less than 10% body weight loss occurred during the treatment period compared to the saline control. Overall, both liposomal formulations appeared to be well tolerated. Body weight and body weight gain appeared to be less affected overall in the LC3-PLDMS-2 and LC4-PLDMS-5 groups than in the doxorubicin HCl treatment group (see FIG. 7A).

LC3-PLDMS-2 and LC4-PLDMS-5 both exhibited improved survival benefit compared to doxorubicin HCl (% ILS values of 26.9, 26.9 days for the 2 PLDMS formulations compared with 21.2, day for doxorubicin as is respectively), reflecting their increased median life span (66 days in each group compared to 63 days in the doxorubicin HCl group). Median TV was decreased relative to the saline control, with volumes of 895.1 and 1123.4 in the mm³ the LC4-PLDMS-5 and LC3-PLDMS-2 groups, respectively on day 52, when the saline control group was terminated due to tumor size. The doxorubicin HCl treatment group at this time point was 1430.3 mm³. Other anti-tumor efficacy measures (listed above), all derived from median tumor volume, consistently showed the improved anti-tumor activity of the two liposomal formulations compared to doxorubicin HCl as well as their relative activity. Tumor growth delay was evident at all the predetermined time points, e.g., at tumor volumes of 250, 500 and 1000 mm³ in the treatment groups.

In summary, both the LC3-PLDMS-2 and LC4-PLDMS-5 formulations showed promising anti-tumor therapeutic efficacy which are higher than equivalent dose of doxorubicin as is in all measured parameters of activity as well as better tolerability.

### Study Design

Mice bearing growing tumor were selected and randomized into 4 groups containing 8 animals in each based on a mean tumor size of ˜100 mm3. G1 animals served as vehicle control and received 5 ml/kg saline whereas G2, G5 and G6 animals received Doxorubicin HCl, LC3-PLDMS-2 or LC4-PLDMS-5, respectively, at a dose of 8 mg/kg. All the animals were dosed intravenously at the dose volume of 5 ml/kg weekly once for two weeks. Body weight, general clinical observations and tumor volume parameters were recorded during the experimental period. Groups reaching median tumor volume of 2500 mm³ were terminated.

For preparation and administration of reference and test item, all the test items are ready to use formulations. The strength of test formulation is 2 mg/ml of doxorubicin HCl in sterile 5 ml vial. 2 ml of reference or test item formulation (i.e. Doxorubicin HCl, LC3-PLDMS-2 and LC4-PLDMS-5) was diluted to 0.5 ml normal saline to achieve 1.6 mg/ml. 100 μL of final test formulation was injected intravenously to 20 g of mouse to achieve the 8 mg/kg dose. Group G1 animals received normal saline at the dose volume of 5 ml/kg.

Median survival time and percent ILS is shown in Table 10 and FIG. 7.

TABLE 10
Effect of treatment on Median Survival Time and % ILS

| | Median Survival | % Increased Life Span |
|---|---|---|
| Treatment | Time (MST) | (% ILS) |
| Vehicle Control, Saline | 52 | 0.0 |
| Doxorubicin HCl | 63 | 21.2 |
| LC3-PLDMS-2 | 66 | 26.9 |
| LC4-PLDMS-5 | 66 | 26.9 |

The body weight of each mouse was recorded at the time of randomization, on alternate days during dosing period and post-dosing period until the end of the study. The observed body weight gain was not more than 7.5% among all the treatment groups by the end of experiment on Day 66, when G5 and G6 showed 4.4% and 5.9% respectively. Additionally no body weight gain was observed in G2. A maximum body weight loss of −8.6% was observed in G2 on day 46 followed by −5.9% in G5, −5.0% in G6 and −1.4% in G1 on 32 as well as on day 34 respectively.

Body weight parameters were statistically analyzed using one-way ANOVA (Dunnett's multiple comparison) and no statistical difference was found in any of the treatment groups in comparison with vehicle control group except in treatment of Doxorubicin HCl (G2) on Day 46, 48, 50 and 52 which showed statistical difference of P<0.05.

Mean body weight and % change in body weight for each group are presented in tables 11 and 12 and graphically in FIGS. 7A and 7C, respectively.

TABLE 11
Mean Body Weight (Unit: g ± SD)

| | Vehicle | | Doxorubicin | | LC3-PLDMS- | | LC4-PLDMS- | |
|---|---|---|---|---|---|---|---|---|
| | control (G1) | | HCl (G2) | | 2 (G5) | | 5 (G6) | |
| Days | Mean | SD | Mean | SD | Mean | SD | Mean | SD |
| 22 | 22.1 | 1.76 | 22.4 | 2.63 | 22.9 | 2.07 | 23.0 | 2.60 |
| 24 | 22.1 | 1.73 | 22.1 | 2.59 | 22.6 | 2.05 | 22.7 | 2.54 |
| 26 | 22.2 | 1.72 | 21.6 | 2.02 | 22.0 | 1.90 | 22.5 | 2.51 |
| 28 | 22.1 | 1.69 | 21.8 | 1.92 | 21.7 | 1.92 | 22.2 | 2.58 |
| 30 | 22.0 | 1.65 | 21.5 | 1.97 | 21.6 | 1.84 | 22.2 | 2.68 |
| 32 | 21.8 | 1.60 | 21.4 | 1.97 | 21.6 | 1.77 | 22.1 | 2.69 |
| 34 | 21.8 | 1.64 | 21.1 | 1.73 | 21.8 | 1.80 | 22.1 | 2.56 |
| 36 | 22.1 | 1.68 | 20.9 | 1.61 | 22.2 | 1.74 | 21.9 | 2.45 |
| 38 | 22.6 | 1.66 | 20.8 | 1.64 | 22.4 | 1.86 | 22.0 | 2.50 |
| 40 | 22.7 | 1.63 | 20.6 | 1.64 | 22.4 | 1.83 | 21.9 | 2.33 |
| 42 | 22.9 | 1.62 | 20.6 | 1.50 | 22.5 | 1.86 | 22.0 | 2.36 |
| 44 | 23.1 | 1.59 | 20.5 | 1.49 | 22.5 | 1.91 | 22.1 | 2.32 |
| 46 | 23.3 | 1.56 | 20.5* | 1.37 | 22.3 | 1.85 | 22.2 | 2.20 |
| 48 | 23.4 | 1.58 | 20.8* | 1.33 | 22.4 | 1.79 | 22.5 | 2.09 |
| 50 | 23.6 | 1.48 | 20.9* | 1.25 | 22.4 | 1.83 | 22.6 | 2.12 |
| 52 | 23.8 | 1.51 | 21.1* | 1.23 | 22.6 | 1.95 | 22.7 | 2.21 |
| 56 | NA | NA | 21.4 | 1.28 | 22.9 | 1.97 | 23.1 | 2.14 |
| 59 | NA | NA | 21.8 | 1.20 | 23.2 | 2.03 | 23.4 | 2.03 |
| 63 | NA | NA | 22.1 | 1.20 | 23.6 | 1.94 | 24.0 | 1.86 |
| 66 | NA | NA | NA | NA | 23.9 | 2.01 | 24.4 | 2.01 |

*= P < 0.05 (One way ANOVA Followed by Dunnett's Multiple comparison Test)

TABLE 12
Mean Percentage Change in Body Weight (Unit: %)

| | Vehicle | Doxorubicin | LC3-PLDMS-2 | LC4-PLDMS-5 |
|---|---|---|---|---|
| Days | control (G1) | HCl-(G2) | (G5) | (G6) |
| 22 | 0.0 | 0.0 | 0.0 | 0.0 |
| 24 | −0.1 | −1.6 | −1.4 | −1.4 |
| 26 | 0.3 | −3.7 | −4.3 | −2.3 |
| 28 | −0.1 | −2.9 | −5.3 | −3.6 |
| 30 | −0.7 | −4.1 | −5.9 | −3.5 |
| 32 | −1.4 | −4.7 | −5.9 | −3.9 |
| 34 | −1.4 | −6.1 | −5.0 | −4.2 |
| 36 | −0.3 | −6.6 | −3.4 | −4.8 |
| 38 | 1.9 | −7.4 | −2.4 | −4.6 |
| 40 | 2.6 | −8.0 | −2.2 | −5.0 |
| 42 | 3.3 | −8.1 | −1.9 | −4.5 |
| 44 | 4.2 | −8.5 | −1.9 | −3.9 |
| 46 | 5.1 | −8.6 | −2.6 | −3.4 |
| 48 | 5.8 | −7.3 | −2.2 | −2.4 |
| 50 | 6.6 | −6.9 | −2.3 | −1.7 |
| 52 | 7.5 | −6.0 | −1.7 | −1.2 |
| 56 | NA | −4.7 | −0.4 | 0.3 |
| 59 | NA | −2.9 | 1.3 | 1.8 |
| 63 | NA | −1.3 | 2.9 | 4.1 |
| 66 | NA | NA | 4.4 | 5.9 |

Median tumor volume was also measured. In particular, tumor volume data and various parameters like median tumor growth inhibition, median % T/C, Log Cell Kill, Tumor volume doubling time, Tumor volume doubling time delay, Tumor Growth Delay and Relative tumor volume were derived and calculated from median tumor volume.

All test item groups (i.e. LC3-PLDMS-2 and LC4-PLDMS-5) and doxorubicin HCl group had decreased median tumor volume (TV) in comparison to the saline control group. On Day 52 (when the saline control group was sacrificed), LC4-PLDMS-5 (G6) had the smallest median tumor volume of 895.1 mm3, followed by a median tumor volume of 1123.4 mm3 in LC3-PLDMS-2 (G5). The vehicle control group (G1) showed median tumor volume of 2556.4 mm3 on Day 52. Doxorubicin HCl treatment group (G2) showed a median tumor volume of 1430.3 mm³ on day 52. After day 52, rest groups were sacrificed accordingly when they reached median tumor volume of 2500 mm3 or above until the end of experimental period. Median tumor volume data are presented in Table 13 and FIG. 7D.

TABLE 13
Median Tumor Volume (Unit: mm³)

| | Vehicle | Doxorubicin | LC3- | LC4- |
|---|---|---|---|---|
| | control | HCl | PLDMS-2 | PLDMS-5 |
| Days | (G1) | (G2) | (G5) | (G6) |
| 22 | 110.2 | 110.9 | 108.9 | 109.2 |
| 24 | 150.9 | 128.6 | 132.0 | 134.7 |
| 26 | 197.8 | 145.8 | 146.1 | 157.9 |
| 28 | 245.5 | 173.8 | 191.3 | 187.9 |
| 30 | 324.9 | 194.7 | 220.6 | 231.1 |
| 32 | 450.2 | 215.5 | 244.6 | 262.1 |
| 34 | 537.6 | 252.9 | 291.0 | 311.9 |
| 36 | 681.7 | 311.1 | 343.2 | 343.7 |
| 38 | 780.2 | 379.4 | 409.5 | 384.8 |
| 40 | 978.6 | 453.8 | 447.5 | 463.9 |
| 42 | 1192.5 | 550.9 | 480.8 | 507.2 |
| 44 | 1405.3 | 673.4 | 563.6 | 559.4 |
| 46 | 1711.6 | 802.4 | 667.5 | 621.2 |
| 48 | 1897.3 | 1006.1 | 803.0 | 706.7 |
| 50 | 2190.8 | 1218.8 | 942.1 | 790.5 |
| 52 | 2556.4 | 1430.3 | 1123.4 | 895.1 |
| 56 | NA | 1799.5 | 1467.1 | 1280.3 |
| 59 | NA | 2192.6 | 1823.2 | 1734.9 |
| 63 | NA | 2699.3 | 2164.1 | 2162.8 |
| 66 | NA | NA | 2724.1 | 2531.1 |

### Log Cell Kill (LCK)

Log cell kill defines the change in tumor size that is directly (linearly) related to the logarithm of the number of cells killed. The maximum log cell kill value of 0.66 was observed in LC4-PLDMS-5 (G6), followed by LC3-PLDMS-2 (G5) with an LCK value of 0.56 and doxorubicin HCL group (G2) with an LCK value of 0.40. Data on Log Cell Kill are presented in Table 14 and FIG. 7E.

TABLE 14
Log Cell Kill (LCK)

| Treatment | Log Cell Kill Value |
|---|---|
| Doxorubicin HCl (G2) | 0.40 |
| LC3-PLDMS-2 (G5) | 0.56 |
| LC4-PLDMS-5 (G6) | 0.66 |

### Mean Tumor Volume Doubling Time (Mean TVDT)

Tumor volume doubling time refers to time taken by tumor to double its volume; it is widely used for quantification of tumor growth rate. In this study, the vehicle control tumor doubled its volume in shortest time, 6 days. In comparison, tumor doubling times were 11, 9.5 and 7 days in the LC4-PLDMS-5 (G6), LC3-PLDMS-2(G5) and doxorubicin HCl groups, respectively.

Data on Mean Tumor Volume Doubling Time are presented in Table 15 and FIG. 7F.

TABLE 15
Mean tumor volume doubling time (Mean TVDT)

| Treatment | Mean TVDT(Days) |
|---|---|
| Vehicle Control, Saline(G1) | 6 |
| Doxorubicin HCl (G2) | 7 |
| LC3-PLDMS-2 (G3) | 9.5 |
| LC4-PLDMS-5 (G4) | 11 |

### Example 12

### Dog Study

### Statement of Purpose/Objectives

The primary objective of this study is to evaluate safety, maximum tolerated dose (MTD), dose limiting toxicities (DLT) and basic pharmacokinetic properties for PLDMS in client-owned dogs (weighing 10 kg), with spontaneous tumors. A secondary objective of the study will be to characterize the frequency and intensity of palmar-plantar erythrodysesthesia (PPE) in dogs with spontaneous tumors treated with PLDMS using standard criteria and comparison to a group of published historical controls receiving pegylated liposomal doxorubicin (Doxil®).

We plan to accomplish the objective of this application by pursuing the following Specific Aims:

Specific Aim 1. Determine the MTD, DLT and Adverse Event (AE) Profile of PLDMS in Client-Owned Dogs with Spontaneous Tumors.

This will be accomplished through the completion of a standard phase I dose-finding trial (3+3 cohort design) which includes assessment of AEs using VCOG-CTCAE v1.1 adverse event characterizations.

### Specific Aim 2. Once the MTD is Established in Aim 1, Determine the Pharmacokinetic Properties of PLDMS in Client-Owned Dogs.

This will be accomplished through the treatment of an expanded cohort (n=6) of additional client-owned dogs treated at the PLDMS MTD (established in Aim 1). Dogs in the expanded cohort will be phlebotomized at time intervals following treatment to establish t½ (h), Cmax (nmol/L), Tmax (h), AUC(0-∞) (nmol/L h).

Specific Aim 3. Determine the Frequency, Intensity and Characteristics of PPE in all Treatment Cohorts, in Particular the Expanded Cohort, and Compare to a Group of Historical Client-Own Dogs Treated with the MTD of Doxil®.

This will be accomplished through the application of a previously established clinical and histopathological cutaneous toxicity scoring system that has been applied to dogs treated with Doxil®.

### Basic Study Design:

### Entry Criteria

Dogs with histologically confirmed measurable tumors of any histology with a likelihood of being responsive to doxorubicin based on the current literature (e.g., lymphoma, carcinoma, soft tissue sarcoma and osteosarcoma).
Any age, gender or breed with satisfactory health.
Any grade and stage of disease.
Dogs must have a Modified Performance Status of 0 (fully active, able to perform at pre-disease level) or 1 (activity less than pre-disease level, but able to function as an acceptable pet).
The client must provide written, informed consent prior to enrolling in the study.

### Exclusion Criteria

Past chemotherapy or radiation therapy in the 3-weeks prior to trial entry.
Body weight ≦10 kg.
Any concurrent disease state that would require additional therapy and, that, in the investigator's opinion, could result in a life expectancy of less than 3 months.
Serum transaminases exceeding 3×ULN.
Serum bilirubin exceeding the reference range.
Serum creatinine exceeding 1.5×ULN.
Neutrophils <2000/mL, platelets <75,000/mL, hematocrit <25%.

### Pretreatment Evaluation

Physical examination
Complete blood count (CBC), serum biochemistry profile, urinalysis (UA)
Tumor biopsy

### Treatment Protocol

### Specific Aim 1:

After informed consent, dogs will receive q3wk dosing of PLDMS according to a standard 3+3 phase I design, beginning with an initial cohort at 0.25 mg/kg i.v (Cohort 1), every 3 weeks for a total of 5 cycles. Dose escalations will be made with 3 dogs per dose level at an escalation level of 0.25 mg/kg per cohort. For this study, a DLT is defined as ≧Grade 3 toxicity (VCOG-CTCAE v1.1) in any AE category except for neutropenia, where a Grade 4 toxicity is dose-limiting. When one dog in a dosing group experiences a DLT, the cohort will be expanded to 6 dogs at that dose level. Escalation to the next higher dose cohort will occur if 0/3 dogs in a cohort experience a DLT or if only 1/6 dogs in an expanded cohort experiences a DLT. If a DLT attributable to treatment is observed in more than 1 dog at a dose level, then the MTD has been exceeded, accrual to that dose level will cease, and dose-escalation will be terminated. The prior dosing cohort will then be expanded to a minimum of 6 dogs and the MTD will be defined as the highest dose level in which no more than 1/6 dogs develops a DLT.

All dogs in a cohort must be observed for at least 3 weeks following initiation of treatment before beginning accrual to the next higher dose level. Five dosing cohorts are planned (cohort 1, 0.25 mg/kg; cohort 2, 0.5 mg/kg; cohort 3, 0.75 mg/kg; cohort 4, 1.0 mg/kg; and cohort 5, 1.25 mg/kg) up to a final dosing cohort of 1.25 mg/kg (the MTD previously established for Doxil® in tumor bearing dogs from previous trials was 1.0 mg/kg). This translates into a likely total of 18-21 dogs, allowing for 2 cohort expansions.

Based on the known AE profile of Doxil®, CBCs, serum biochemistry profiles, UAs, physical examinations (including body weight) and quality of life questionnaires will be completed to assess AEs at time points outlined in table 1. Clients will be responsible for initial screening which will include all of the above assessments. Expected and unexpected AEs will be reported and likely attribution assigned according to VCOG-CTCAE v1.1.

TABLE 16
Assessment Schedule

| | Days in Study | | | | | | | | | | |
|---|---|---|---|---|---|---|---|---|---|---|---|
| Evaluation | 0 | 7 | 21 | 28 | 42 | 49 | 63 | 70 | 84 | 91 | 105 |
| Physical | X | X | X | X | X | X | X | X | X | X | X |
| Examination | | | | | | | | | | | |
| CBC | X | X | X | X | X | X | X | X | X | X | X |
| Biochemistry | X | | X | | X | | X | | X | | X |
| Profile | | | | | | | | | | | |
| Urinalysis | X | | X | | X | | X | | X | | X |
| Tumor Biopsy | X | | | | | | | | | | |
| Tumor | X | | X | | X | | X | | X | | X |
| Measurements | | | | | | | | | | | |
| Quality of Live | X | | X | | X | | X | | X | | X |
| Assessment | | | | | | | | | | | |
| Cutaneous | X | | X | | X | | X | | X | | X |
| Toxicity Score | | | | | | | | | | | |
| Dermal Punch | X | | X | | X | | X | | X | | X |
| Biopsy | | | | | | | | | | | |
| RECIST | X | | X | | X | | X | | X | | X |
| measure | | | | | | | | | | | |
| PLDMS | X | | X | | X | | X | | X | | |
| treatment | | | | | | | | | | | |
| PK sampling | X | | | | | | | | | | |

### Specific Aim 2

An expanded cohort (n=6) of additional client-owned dogs treated at the PLDMS MTD (established in Aim 1) will be phlebotomized at 6 time intervals following treatment to establish t½ (h), Cmax (nmol/L), Tmax (h), and AUC(0-∞) (nmol/L h). Dogs in the expanded cohort will also receive 5 total cycles of PLDMS.

### Specific Aim 3

The frequency, intensity and characteristics of PPE in dogs in all treatment cohorts will be determined through the application of a previously established clinical and histopathological cutaneous toxicity scoring system that has previously been applied to dogs treated with Doxil® to assess PPE. This includes a clinical assessment scale and pre-treatment and post-treatment dermal biopsies as outlined in Table 1.

The 6 dogs in this expanded cohort plus the 6 dogs in the phase I MTD cohort (n=12) will be compared to a group of previously published historical client-owned dogs treated with the MTD of Doxil®. While this is likely underpowered, inferences as to PPE intensity will be drawn and additional dogs treated as deemed necessary by the study sponsor.

Antitumor activity: While not a primary endpoint of phase I trials, tumor measurements will be performed prior to initiation of therapy and at each subsequent recheck. Standard RECIST v1.1 criteria for the assessment of solid tumors will be applied.

Continuation of treatment: Dogs in each treatment cohort will receive a minimum of 2 treatments (unless toxicity prohibits continuation) and continued therapy will occur until progressive disease (by RECIST v1.1 criteria) or 5 total cycles is concluded, whichever occurs first.

Patient Numbers: Based on the starting dose and the dose escalation scheme (0.25 mg/kg increments), a maximum of 5 cohorts will be evaluated, as the 4^{th} cohort would be at the current MTD for Doxil®. With 3 dogs per cohort and 6 additional dogs for expansion of 2 cohorts being possible, the total of 21 dogs is justifiable for specific aim 1. An additional 6 dogs are required for specific aim 2, bringing the likely total to 27 dogs.

Time to completion: Based on 3-week observational periods for each cohort and a maximum of 5 cohorts, it is anticipated that the trial will be completed within 5-6 months.

### Summary of Results:

The study will help determine the maximally tolerated dose as grade to AEs (just shy of dose limiting) for example higher dosing cohort. Assessment of PPE cutaneous toxicity and comparison to Doxil® (Aim 3) should begin to provide clinically significant data as we are now at doses and near cycle numbers known to produce PPE in Doxil® treated dogs. Regarding Aim 2, PK samples will be stored and collected in dose-appropriate cases necessary to complete this aim. Regarding anti-tumor activity of test article, early results suggest equivalent activity when compared to similar populations treated with free doxorubicin or Doxil®.

1-16. (canceled)

17. A liposome comprising
(i) an entrapped amphipathic weak base; and
(ii) an entrapped alkyl sulfonate salt or ion.

18. The liposome of claim 17, wherein the alkyl sulfonate is an ammonium alkyl sulfonate.

19. The liposome of claim 17, wherein the alkyl sulfonate is selected from the group consisting of methanesulfonate, ethanesulfonate, 3-hydroxypropane-1-sulfonate, 2-hydroxyethanesulfonate, 1,3-dihydroxy-2-(hydroxymethyl)-2-propanesulfinic acid, 2-hydroxy-1-(2-hydroxyethoxy)-2-propanesulfonic acid, and 4-hydroxy-3,3-bis(hydroxymethyl)-1-butanesulfonic acid.

20. The liposome of claim 17, wherein the amphipathic weak base comprises doxorubicin, vincristine and/or topotecan.

21. The liposome of claim 17, wherein the liposome is between 20 and 2000 nm in diameter.

22. The liposome of claim 17, wherein the liposome is pegylated.

23. The liposome of claim 17, wherein said amphipathic weak base is selected from the group consisting of doxorubicin, vincristine and topotecan; and wherein said alkyl sulfonate is an ammonium alkyl sulfonate.

24. The liposome of claim 23, wherein said ammonium alkyl sulfonate is selected from the group consisting of methanesulfonate, ethanesulfonate, 3-hydroxypropane-1-sulfonate, 2-hydroxyethanesulfonate, 1,3-dihydroxy-2-(hydroxymethyl)-2-propanesulfinic acid, 2-hydroxy-1-(2-hydroxyethoxy)-2-propanesulfonic acid, and 4-hydroxy-3,3-bis(hydroxymethyl)-1-butanesulfonic acid.

25. A composition comprising a liposome according to claim 17.

26. A composition comprising a liposome according to claim 23.

27. A composition comprising a liposome according to claim 24.

28. The composition of claim 25, further comprising a chemotherapeutic agent.

29. A method of making liposomes according to claim 17, the method comprising:
(i) preparing a suspension of liposomes, each liposome in the suspension having at least one internal aqueous compartment that contains an alkyl sulfonate at a first concentration, the liposomes suspended in an external bulk medium comprising the alkyl sulfonate at the first concentration;
(ii) introducing the weak amphipathic base to the suspension; and
(iii) reducing the concentration of the alkyl sulfonate in the external bulk medium to second concentration, wherein the second concentration is lower than the first concentration, establishing an ion concentration gradient across lipid bilayers of the liposomes such that the weak amphipathic base is transported to the inside of the liposomes.

30. The method of claim 29, wherein the concentration of alkyl sulfonate in the external bulk medium is reduced by dilution, dialysis, diafiltration and/or ion exchange.

31. The method of claim 29, wherein at least 90% of the amount of the weak amphipathic base added to the suspension is transported to the inside of the liposomes.

32. A method of treating cancer, the method comprising administering to a subject in need thereof a composition according to claim 25.

33. A method of treating cancer, the method comprising administering to a subject in need thereof a composition according to claim 27.

34. The method of claim 32, further comprising administering an additional chemotherapeutic agent to the subject.

35. A method of reducing one or more side effects associated with administration of a liposomal amphipathic weak base, the methods comprising administering a liposome according to claim 17 to a subject in need thereof.

36. The method of claim 35, wherein the one or more side effects is selected from: oral, intestinal and/or ocular mucositis, asthenia, sleep disruption and palmar-plantar erythrodysesthesia (PPE).
